SEQUENCE LISTING (9 sequences)

SEQ ID NO: 1 - DNA, 1675 bp, Homo sapiens
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 2 - DNA, 1604 bp, Rattus norvegicus
cctgcagggcccactagtctgtaagctggaagtctggcagtgctgagctggccaaccccctcaggacctcctccttgtgcccactgaatgactcaccttggcatagacataatggtcaggggcgggcacacagcctgattcccgctgcactccaggcccccttcaatgctttccgagaagtccattgagctgggagcttgtactgcaccaagggctgacatcctggcagccagggatgaaagcagcccatggggctacccttgccgtatgcctcactggcggcagagaacaaggctctattcagcaaatgccctggagtagacaccagaagtccaagcatgggcagaggaaggcaggcgttgggggctggaggggagcagagctgtctgttttccagaagcccaagggtacagatggcgcctgggggggaactgagtggaggggatagatgggcctgagatctcaaacatcaacagcctcctccccaccaacgatgaaggtggaggttggtttcccagacctacatatcccccagagacctggtgtatgaaaattcaaaggaggtaagtctcctgagagaacggggggctcacaaatgaagccagctgtcttaccctatcaggacctacgtgcattccttctgtcctgccccctaaacacacagccagaggctcaaattgattctggagtcacaaagggggcttgaaaccccagccccccactcctgaactccaggaatgagaagatagtattggaggggttcagaggagagggctctgcacatctgttgagaatgggggtcccaggagagtgtaatttaggctgatcccggaggaagggaataggctcttcaagatcctagcatctcacaggcccacagagaagttcagagttggggcagccctggcttacaggctctaagaactggaggcagtttacccaacccagctgtgtgcatgctgtccctctctctgtctctgtctgtctctctctgtctctgtctctctgtgtgtgtgtgtgtgtgctcacacacgtgtgtgtttatcacacaaatgttcatgtgtgtgtacatacatgtgttgaggccagaggtcaacctcagacactgttgacttggttgtatgagataacatttccccctgggacctgggatttgccaattagtgtgacccaggaagcctacttattttcattcctcagcactgcagttacaagtatgcactgtcaaaccaggcctttttttttttttttttccaaaccaggccttttgtattcgctctgtggctagaacttgggtctccatgcttgacaggcaagcgatttatggactaagctgtttcctcggccctctcttgacccatttaccagaaatggggtttccttgatcaatggttaagccaggctggtgttcccaggaaacccttgactctgggtacagtgaccttggtggggtgagaagagttctctccatagctgggctggggcccagctccaccccctcaggctattcaatggggtgctgccaggaagtcaggggcagatccagtccagcccgtccctcaataaaggccctgacatcccaggagccagcagaagcagggcat

SEQ ID NO: 3 - DNA, 21 bp, Homo sapiens
cagctatgccagcatcttgcc

SEQ ID NO: 4 - DNA, 22 bp, Homo sapiens
agccaagctcagacggatccga

SEQ ID NO: 5 - DNA, 22 bp, Homo sapiens
tgagctacagtgcttcatctca

SEQ ID NO: 6 - RNA, 24 nt, artificial
  synthetic primer
ggcaagaugcuggcauagcugaaa

SEQ ID NO: 7 - RNA, 25 nt, artificial
  synthetic primer
ucggauccgucugagcuuggcuaaa

SEQ ID NO: 8 - RNA, 22 nt, artificial
  synthetic primer
agaugaagcacuguagcucaaa

SEQ ID NO: 9 - DNA, 31 bp, artificial
  synthetic primer
ccgcctgggaataccgggtgctgtaggcttt

(19) United States
(12) Patent Application Publication
(10) Pub. No.: US 2011/0098346 A1
(43) Pub. Date: Apr. 28, 2011

(54) NUCLEIC ACID MOLECULE AND METHOD OF TARGETING GENE EXPRESSION TO GLIOMAS

(75) Inventors: Shu Wang, Singapore (SG); Chunxiao Wu, Singapore (SG)

(73) Assignee: Agency for Science, Technology and Research

(21) Appl. No.: 12/993,787

(22) PCT Filed: Nov. 4, 2008
(86) PCT No.: PCT/SG2008/000423

§ 371 Date: Nov. 19, 2010

Related U.S. Application Data
(60) Provisional application No. 61/071,814, filed on May 19, 2008.

(51) Int. Cl. A61K 31/7088; C12N 15/63; C12N 15/85; C12N 5/10; A61P 35/00

(52) U.S. Cl. 514/44 R; 435/320.1; 435/455; 435/325

(57) ABSTRACT

There is presently provided a nucleic acid molecule comprising a glial-specific promoter; a coding sequence for a transgene; and a plurality of miRNA target sites. Each miRNA target site binds an miRNA that is down-regulated in .a glioma cell compared to a normal glial cell, and the glial-specific promoter and the plurality of miRNA target sites are both operably linked to the coding sequence for the transgene.

### CROSS-REFERENCE TO RELATED APPLICATION

This application claims benefit of, and priority from, U.S. provisional patent application No. 61/071,814, filed May 19, 2008, the contents of which are incorporated herein by reference.

### FIELD OF THE INVENTION

The present invention relates generally to nucleic acid molecules, expression vectors and methods of directing gene expression in gliomas.

### BACKGROUND OF THE INVENTION

Specific gene expression in a selected cell or tissue type can be achieved by targeted gene delivery through the use of ligand associated delivery vectors that bind, via the ligands, to cell surface receptors that are unique to the target cells. Specific gene expression can also be achieved by targeted transcription through the use of cell-specific promoters and enhancers. Cell-specific promoters are one of the primary means through which specialized cellular functions are limited to a particular differentiated cell type. The ability of these promoters to direct transcription of associated genes is regulated by the intracellular concentrations and activities of transcription factors in a specific type of cells.

Cell-type specific transgene expression using a cell-specific cellular promoter to restrict transgene expression in targeted tissues is a useful technique in biological studies to investigate cellular functions of a gene, as well as in medical applications to eliminate side effects caused by off-target expression of a therapeutic gene.

Currently, tissue- or cell-type specific promoters are employed to control specificity of expression of a transgene. This strategy is especially appealing to expression of a suicide gene in cancer treatment, which employs either toxic genes or genes encoding enzymes that turn prodrugs into toxic compounds.

Previous efforts to use tissue- or cell-specific promoters or tumour selective promoters in expression of suicide genes have met with mixed success, suffering from the tightness of such promoters in restricting therapeutic gene expression tightly to tumour cells (Harrington et al, 2000; Robson & Hirst, 2003; Saukkonen and Hemminki, 2005). This is consistent with the notion that defining tissue specificity is not straightforward, as the human genome is pervasively transcribed (Yang et al., 2005; Birney et al., 2007). Moreover, tissue-specific genes may not be truly specific under different physiological conditions and a cellular promoter may exhibit altered induction patterns due to the altered expression of endogenous cis and trans regulatory elements under different pathological conditions (Harrington et al, 2000; Robson & Hirst, 2003; Saukkonen and Hemminki, 2005; Stoff-Khalili et al., 2008). Hence, additional control strategies may be necessary to attain desired tumour selectivity for cancer treatment using suicide gene expression.

Thus, this approach may be hindered by significant off-target expression under certain conditions. For example, while a cell-specific promoter-mediated toxic gene expression can eliminate tumour cells derived from the specific cell type in which the promoter functions, such an approach would also kill healthy cells as a result of expression from the cellular promoter in those cells.

Tissue-specific promoters and tumour-selective promoters have been tested with suicide gene expression to minimize killing effects on non-target normal cells. Distinguishing between a tissue-specific promoter and a tumour-selective promoter relies on the relative activity of the promoter in normal and tumour tissues and the dividing line between them is often blurred (Harrington et al., 2000).

In addition to the above drawbacks, the unique characteristics of the central nervous system (CNS), the most sophisticated organ in the body, present several obstacles to successful tissue- or cell-targeted gene expression. The cell types found within the CNS are very diverse, many of which are critical to physiological functions and highly sensitive to any change in gene expression. The use of selective targeting strategy to control expression of therapeutic genes, for example trials employing expression of suicide genes often involve direct intratumoural injection of vectors expressing a toxic suicide gene, thus confining therapeutic gene expression to the tumour tissue. However, since natural infection spectrums of most of viral vectors are not confined to the tumour tissue, potential leakage of intratumourally injected vectors into normal tissues is a concern and might cause collateral damage to healthy cells. Although such damage is tolerable in certain organs, it might have severe consequences in a sensitive organ like the brain.

These particular challenges require development of specific approaches to expressing transgenes within cells of the CNS, including gene expression that is restricted to a particular type of CNS cells, thus ensuring therapeutic effects in the desired cells and limiting side effects caused by gene expression in non-target CNS cells.

Gliomas are one of the most common types of primary brain tumours. Gliomas can be highly invasive, very aggressive, and may be one of the most incurable forms of human cancers (Ciafre et al., 2005; Pulkkanen and Yla-Herttuala, 2005). Gliomas originate from glial cells, predominantly from astrocytes, and are graded from I to IV with increasing level of malignancy. Grade IV gliomas, also named glioblastoma multiforme (GBM), comprise nearly half of gliomas and are the most frequent primary brain tumours in adults. Conventional treatment of gliomas such as surgery, gamma-irradiation, and chemotherapy are ineffective against gliomas, evidenced by poor prognosis for glioma patients with a mean survival time of less than one year after diagnosis.

Investigational therapies such as targeted gene expression remain one of the most promising approaches to treat gliomas. Such approaches require the development of a selective targeting strategy that can kill tumour cells specifically while leaving non-target healthy cells of the CNS intact.

Thus, the full potential of treating gliomas using tumour-specific expression of therapeutic suicide genes would benefit from development of approaches that permit more precise control of suicide gene expression within glioma cells without targeting healthy cells of the CNS.

### SUMMARY OF THE INVENTION

In one aspect, the present invention provides a nucleic acid molecule comprising: a glial-specific promoter; a coding sequence for a transgene; and a plurality of miRNA target sites; wherein each miRNA target site binds an miRNA that is down-regulated in a glioma cell compared to a normal glial cell, and wherein the glial-specific promoter and the plurality of miRNA target sites are both operably linked to the coding sequence for the transgene.

The glial-specific promoter may be an astrocyte-specific promoter, including a glial fibrillary acidic protein promoter.

The plurality of miRNA target sites may comprise at least one has-miR-31, has-miR-127 or has-miR-143 target site. In certain embodiments, the plurality of miRNA target sites comprises: at least one has-miR-31 target site, at least one has-miR-127 target site and at least one has-miR-143 target site; or at least two has-miR-31 target sites, at least two has-miR-127 target sites and at least two has-miR-143 target sites.

The transgene may encode a gene product that induces direct killing of the glioma cell, an immunomodulation protein, a cytotoxin, an angiogenesis inhibitor protein, a tumour suppressor protein, a suicide protein, an apoptotic protein, an anti-angiogenic protein or an antibody. For example, the transgene may encode HSV-tk or DT-A.

In another aspect, the invention provides an expression vector comprising a nucleic acid molecule of the invention, as described herein.

The expression vector may be a baculoviral vector.

In yet another aspect, the present invention provides a method of expressing a transgene in a glioma cell comprising: transfecting a glioma cell with an expression vector of the invention.

The glioma cell may be an in vitro cell, an ex vivo cell explanted from a subject or an in vivo cell in a subject.

In a further aspect of the present invention, there is provided a transgenic cell comprising an expression vector of the invention.

In yet a further aspect of the present invention, there is provided a pharmaceutical composition comprising an expression vector of the invention.

In still a further aspect of the present invention, there is provided a kit comprising an expression vector of the invention, and instructions for expressing a transgene in a glioma cell.

Other aspects and features of the present invention will become apparent to those of ordinary skill in the art upon review of the following description of specific embodiments of the invention in conjunction with the accompanying tables and figures.

### BRIEF DESCRIPTION OF THE DRAWINGS

In the tables and figures, which illustrate, by way of example only, embodiments of the present invention:

Table 1: Sequence of primers used to construct particular embodiments of the expression vector of the invention.

FIG. 1: Selection of miRNAs for regulation of transgene expression. (a) Identification of miRNAs with high copy numbers in normal human astrocytes and significantly downregulated in glioblasmtoma cells. The copy number of different miRNAs in normal astrocytes was estimated based on the average signal intensity of 10 astrocyte samples from 10 microarray assays and the absolute copy number of miR-31 quantified using real-time PCR. Two hundred thirty-one miRNAs that are expressed both in normal astrocytes and glioblasmtoma cells are included in the figure. The dots of three selected miRNAs are decorated in blue. (b) miRNA expression analysis by RT-qPCR. The expression of three selected miRNAs, miRNA-31, miRNA-127 and miRNA-143, in U87 cells and NHA were quantified. miRNA copy numbers were calculated based on a standard curve generated using a synthetic miR-31 RNA oligonucleotide.

FIG. 2: Combinatorial effects of GFAP promoter and miRNA regulation on luciferase transgene expression. (a) Schematic representation of the expression cassettes containing an enhanced, astrocyte-specific GFAP promoter and miRNA target sequences. pFastBac plasmid (pFB) vectors with a GFAP promoter and different miRNA target sequences were constructed and tested first. CMV E/GFAP: a hybrid promoter constructed by appending a 380-be fragment of the enhancer of human cytomegalovirus immediate-early gene 5′ to the human GFAP promoter. luc: luciferase gene. miRNA target sequences as detailed in Table 1 were inserted into the 3′ UTR regions of the luc gene. pA: polyA signal. The pFastBac plasmids without miRNA target sequence insertion and with a mix9F sequence were used as shuttle vectors to generate baculoviral vector BV-luc-ctrl and BV-luc-mix9F respectively. (b) Screening miRNA target sequences that regulate transgene expression in normal human astrocytes, but not in glioblastoma cells. The pFB vectors with different miRNA target sequences were used for transfection of U87 cells and normal human astrocytes. Luciferase gene expression was analyzed 1 day after transfection. The results are shown as the percentage of control pFB without miRNA target sequence insertion. (c) In vitro luciferase transgene expression mediated by baculoviral vectors. Cells were transduced with increased MOI from 2 to 50 and Luciferase gene expression was analyzed 1 day after transduction. The results from BV-luc-mix9F are shown as the percentage of BV-luc-ctrl. (d) Luciferase transgene expression in the brain mediated by baculoviral vectors. Two days after BV-luc-ctrl or BV-luc-mix9F injection into the striatum of the mouse brain, brain tissues were collected for luciferase activity assays. The results are expressed in relative light units (RLU) per brain. Columns, means (n=4); bars, SD. *: p<0.05.

FIG. 3: Selective cellular effects of BV-HSVtk. (a) Western blot analysis of HSVtk expression in BV-HSVtk-ctrl- or BV-HSVtk-mix9F-transduced human neurons, astrocytes and U87 glioblastoma cells and mouse brain. (b) Cell viability of BV-HSVtk-ctrl- or BV-HSVtk-mix9F-transduced human neurons, astrocytes and U87 glioblastoma cells after GCV treatment for 2 days. (c) GFAP immunostaining of brain sections from BV-HSVtk-ctrl- and BV-HSVtk-mix9F-injected mice show the protection of GFAP-positive astrocytes by incorporating miRNA targeting sequences into baculoviral expression cassette. STR: Striatum. (d) Western blot analysis of GFAP expression in the BV-HSVtk-ctrl- and BV-HSVtk-mix9F-injected mouse brain show the protection of GFAP-positive astrocytes by incorporating miRNA targeting sequences into baculoviral expression cassette.

FIG. 4: BV-HSVtk-ctrl and BV-HSVtk-mix9F displayed same therapeutic effects on glioblastomas. (a) Two representative glioblastoma-bearing mice injected with BV-HSVtk-ctrl (right brain) and BV-HSVtk-mix9F (left brain). A single baculovirus injection was given to each of human U87 glioblastoma xenografts in the brain 7 days after tumour inoculation, followed by i.p. GCV injection for 5 days. Bioluminescence images are shown 0, 2 and 5 days after baculovirus/GCV injection. The bioluminescence signals from the tumour cells reduced to a background level after GCV injection for 5 days, but remained after PBS injection for 5 days. (b) Quantitative analysis of bioluminescence signals from 4 animals. Note that the changes of bioluminescence signals in BV-HSVtk-mix9F-injected tumour xenografts have the same tendency as the changes in BV-HSVtk-ctrl-injected tumour xenografts. (c) Representative pictures of brain sections show U251 xenografts. Left: Five days after GCV injection, only small tumours (arrows) were found in the tissue sections remote from the virus injection site. Right: In the control brain without GCV injection, large and grossly visible tumour masses, with tumour dissemination in the ventricle, were found.

### DETAILED DESCRIPTION

The nucleic acid molecules, expression vectors, methods and uses described herein combine the use of tissue-specific promoter and differential expression of microRNA (miRNA) to preferentially direct gene expression within a glioma cell.

To develop the present nucleic acid molecules, expression vectors, methods and uses, a microarray was used to determine differential miRNA expression in healthy astrocytes and in glioblastoma cells. miRNA target sites for miRNAs that are enriched in healthy astrocytes but down-regulated in glioma cells are used together with an glial-specific promoter to target preferential expression of a gene such as a therapeutic transgene in gliomas, while reducing expression in glial cells, including astrocytes.

Briefly, miRNAs are a recently discovered class of gene regulators that are abundant, endogenous small non-coding single-stranded RNAs of approximately 19-25 nucleotides involved in down-regulation of gene expression in both plants and animals.

miRNAs are initially transcribed by RNA polymerase II from different genomic locations as primary-miRNAs (pri-miRNAs). Pri-miRNAs contain hairpin-loop domains that fold back to form specific stem-loop secondary structures. The hairpin-loop region is cleaved in the nucleus by an endonuclease in complex with a double-stranded RNA-binding protein to liberate a precursor of about 60- to 70-nucleotides (pre-miRNA). The pre-miRNAs are then actively transported from the nucleus to the cytoplasm, where they are cleaved by an RNase. Cleavage results in a mature product of 19-24 nucleotide duplex, which is subsequently unwound by a helicase. The mature miRNAs are asymmetrically incorporated into an effector complex called the RNA-induced silencing complex (RISC) which negatively regulates genes containing sequences targeted by the miRNA.

After base pairing to mRNAs, miRNAs mediate post-transcriptional gene repression through induction of deadenylation, decay of target mRNAs, and/or repression of protein production by inhibition of translation initiation, block of elongation and/or degradation of nascent peptides (Filipowicz et al., 2008). Most animal miRNAs bind to multiple, partially complementary sites in the 3′ untranslated region (3′ UTR) of an mRNA transcript. The complementarity is usually restricted to the first 2-8 bases of the 5′ end of the mature miRNA sequence. The fate of a target mRNA is determined by the extent of base-pairing between the miRNA and its target. Perfect or near-perfect complementarities between miRNA and its target 3′ UTR results in RISC-induced mRNA cleavage, whereas imperfect base matching results mainly in translational silencing of the target without strongly affecting the mRNA levels.

To date, approximately 540 miRNA genes have been indentified in the human genome (http://microrna.sanger.ac.uk). These miRNAs regulate 10 to 30% of all protein-coding genes in the human genome (John et al., 2004; Lewis et al., 2005). A single miRNA might regulate 100 to 200 genes and each gene can be targeted by multiple miRNAs, suggesting a complex regulatory network between miRNAs and their targets (John et al., 2004; Lewis et al., 2005).

Studies have been done to identify the tissue expression patterns of miRNAs and the role of these small RNAs in organ development. Some miRNAs have been found to be expressed widely in the human body, whereas others display restricted expression in development stage-, tissue-, or cell type-specific manners. miRNAs control almost every cellular process investigated so far, including proliferation, stem-cell division, developmental timing, apoptosis, differentiation and metabolism (Bushati & Cohem, 2007; Kloosterman & Plasterk, 2007).

The brain expresses the largest proportion of tissue-specific miRNAs and accumulating evidence points towards widespread post-transcriptional regulation in the brain, with the demonstrated regulatory effects of brain-specific miRNAs in controlling neuronal development, synaptic plasticity, and possibly long-term memory storage (Krichevsky et al., 2003; Babak et al., 2004; Sempere et al., 2004). Consequently, impaired miRNA expression in the brain may lead to loss of differentiation (or de-differentiation), thus resulting in the occurrence of brain tumours (Esquela-Kerscher and Slack, 2006; Rosenfeld et al., 2008). In tumour tissues, miRNA profiling is informative in classifying human cancers, with most of miRNAs in primary tumour tissues and cancer cell lines being down-regulated compared with normal tissues (Lu et al., 2005). Consistent with the notion that miRNA expression is closely linked to differentiation, poorly differentiated tumours have lower global levels of miRNA expression and global expression of miRNAs increases with differentiation of the tumour cells (Lu et al., 2005).

Several studies have used reporter systems to track miRNA expression in situ (Mansfield et aI., 2004; Zhao et al., 2005), however, the degree and robustness of miRNA mediated suppression was less examined. Recent studies by Brown and colleagues demonstrated that endogenous miRNAs can be broadly exploited to regulate transgene expression in different cell types (Brown et al., 2006; 2007). However, the multiplicity and cooperativity of miRNA regulation pose big challenge for selecting endogenous miRNAs for stringent control of transgene expression.

The present nucleic acid molecules, expression vectors, methods and uses are predicated on the concept that the use of a tissue-specific promoter could increase transgene expression preferentially in a specific type of tissues or cells, while the additional use of miRNA regulation could provide another layer of control to differentiate transgene expression between tumour and normal cells of the same lineage.

miRNA activity results in de-targeting rather that targeting of gene expression in cells in which the miRNA is active. This mechanism functions at the post-transcriptional level and offers an approach for overcoming the limitations of current gene delivery systems using tissue-specific promoters, including reducing off-target expression resulting from positional effects of insertion and/or imperfect reconstitution of a tissue-specific promoter. By preventing transgene expression in normal glial cells while permitting high levels of expression in glioma cells, the combination of miRNA regulation and tissue-specific promoter control used in the present nucleic acid molecules, expression vectors, methods and uses may serve to enhance the efficacy and specificity of gene expression in gliomas. Thus, the presently described nucleic acid molecules, including expression vectors may be useful to direct gene expression in treatment of gliomas.

Thus, there is presently provided a nucleic acid molecule comprising a glial-specific promoter, a coding sequence for a transgene and a plurality of miRNA target sites. Each of the miRNA target sites is capable of binding an miRNA that is down-regulated in a glioma cell compared to a normal glial cell. The glial-specific promoter and the plurality of miRNA target sites are both operably linked to the coding sequence for the transgene, thus regulating the expression of the transgene.

As used herein, a “glial cell” or “glia” refers to a non-neuronal cell or cells found in the central nervous system which act as support for neuronal cells, which do not conduct electrical impulses and which play a role in repair and regeneration of nervous tissue. Glial cells include astro-glial cells or astrocytes, which are star-shaped glial cells and which may be fibrous astrocytes or protoplasmic astrocytes. Protoplasmic astrocytes are found mainly in the grey matter while fibrous astrocytes occur mainly in the white matter of the brain and spinal cord. Glial cells also include microglial cells and either oligodendrocytes (in the central nervous system) or Schwann cells (in the peripheral nervous system). A “cell derived from a glial cell” refers to a cell which is derived from, originated from or is differentiated from a glial cell, including an astrocyte, and includes neoplastic and tumour cells, including gliomas and astrocytomas, including low grade and high grade astrocytomas, and may be any such cell, including a mammalian cell, for example a human cell, a rat cell or a mouse cell. A “normal” glial cell, including a normal astrocyte, refers to a glial cell that is not cancerous, tumourous, transformed or neoplastic, in contrast to a glioma cell.

The term “cell” or “cells” refers to a single cell, as well as a plurality of cells, a culture of cells, a growth of cells, a population of cells or a cell line, and may be in vitro or in vivo, where context permits, unless otherwise specified. In vitro cells include ex vivo cells explanted from a subject. Simlarly, reference to “cells” also includes reference to a single cell where context permits, unless otherwise specified.

As will be understood, a promoter or a promoter region is a nucleotide sequence located upstream of a coding region of a gene that contains at least the minimal necessary DNA elements required to direct transcription of the coding region, and typically includes a site that directs RNA polymerase to the transcription initiation start site and one or more transcription factor binding sites. A promoter, including a native promoter may include a core promoter region, for example containing a TATA box and an initiator sequence, and it may further include a regulatory region containing proximal promoter elements outside of the core promoter that act to enhance or regulate the level of transcription from the core promoter, including enhancer elements normally associated with a given promoter.

“Glial” describes something that is of, related to, or comprises, glial cells. Similarly, “astro-glial” describes something that is of, related to, derived from, or comprises, astro-glial cells or astrocytes. The term “glial-specific” refers to something that is found, or an activity that occurs, in glial cells or in cells derived from glial cells but is not found in or occur in, or is not found substantially in or occur substantially in, non-glial cells or in cells not derived from glial cells, for example neuronal cells or cells not found in the central nervous system.

The nucleic acid molecule includes a glial-specific promoter. A glial-specific promoter is a promoter that controls expression of genes that are uniquely or predominantly expressed in glial cells or in cells derived from glial cells, including glioma cells and astrocytes. A glial-specific promoter directs expression of a gene in glial cells or in cells derived from glial cells, but does not substantially direct expression of that same gene in other cell types, for example neuronal cells, thus having glial-specific transcriptional activity. In some instances there may be some low level expression in other cell types, but such expression is substantially lower than in glial cells, for example about less than 1% or about 1%, 2%, 3%, 5%, 10%, 15% or 20% of the expression levels in glial cells.

Such a promoter may be a strong promoter or it may be a weak promoter, and it may direct constitutive expression of a gene in a glial cell or a cell derived from a glial cell, or it may direct expression in response to certain conditions, signals or cellular events. For example, the promoter may be an inducible promoter that requires a particular ligand, small molecule, transcription factor or hormone protein in order to effect transcription from the promoter.

The promoter may be a hybrid promoter, containing elements from a native glial-specific promoter in combination with elements from another promoter, for example a viral promoter such as for example the enhancer element of human cytomegalovirus immediate early gene. It will be appreciated that any hybrid promoter used should retain glial-specificity.

In certain embodiments, the glial-specific promoter includes the JC virus early promoter (Kim, S. Y., et al., J Virol. (2003) 77:3394-401), the myelin basic protein promoter (Wei, Q., et al., Gene. (2003) 313:161-7), or the S100beta promoter (Namihira, M., et al., FEBS Lett. (2004) 572:184-8).

In one embodiment, the glial-specific promoter includes the promoter region of glial fibrillary acidic protein (GFAP) gene, which is specific to astrocytes and cells derived from astrocytes. The GFAP promoter may be any promoter that directs expression of glial fibriallary acid protein. In particular embodiments, the GFAP promoter comprises the 2.2 kb 5′ region flanking the human GFAP gene.

GFAP is an intermediate-filament protein, expressed exclusively in cells of glial origin (Eng, 1985). The protein has been used as pathological marker of astrocytic malignancies (Rutka et al., 1997). Although the GFAP protein is not detectable in man_{y g}lioblastoma multiforme tumours due to hypermethylation of the GFAP promoter, the promoter in a gene transfer vector is nevertheless active in glioma cells (Condorelli et al., 1994; Fukuyama et al., 1996). Hence, the GFAP promoter has been extensively tested for selective transgene expression in glioma cells (Chen et al., 1998; McKie et al., 1998; Wang et al., 2006; Horst et al., 2007).

In particular embodiments, the glial-specific promoter region comprises, consists essentially of or consists of the sequence of the human GFAP promoter or the rat GFAP promoter, as shown below:

Human GFAP promoter [SEQ ID NO: 1]:

GTCTGCAAGCAGACCTGGCAGCATTGGGCTGGCCGCCCCCCAGGGCCTC
CTCTTCATGCCCAGTGAATGACTCACCTTGGCACAGACACAATGTTCGG
GGTGGGCACAGTGCCTGCTTCCCGCCGCACCCCAGCCCCCCTCAAATGC
CTTCCGAGAAGCCCATTGAGTAGGGGGCTTGCATTGCACCCCAGCCTGA
CAGCCTGGCATCTTGGGATAAAAGCAGCACAGCCCCCTAGGGGCTGCCC
TTGCTGTGTGGCGCCACCGGCGGTGGAGAACAAGGCTCTATTCAGCCTG
TGCCCAGGAAAGGGGATCAGGGGATGCCCAGGCATGGACAGTGGGTGGC
AGGGGGGGAGAGGAGGGCTGTCTGCTTCCCAGAAGTCCAAGGACACAAA
TGGGTGAGGGGACTGGGCAGGGTTCTGACCCTGTGGGACCAGAGTGGAG
GGCGTAGATGGACCTGAAGTCTCCAGGGACAACAGGGCCCAGGTCTCAG
GCTCCTAGTTGGGCCCAGTGGCTCCAGCGTTTCCAAACCCATCCATCCC
CAGAGGTTCTTCCCATCTCTCCAGGCTGATGTGTGGGAACTCGAGGAAA
TAAATCTCCAGTGGGAGACGGAGGGGTGGCCAGGGAAACGGGGCGCTGC
AGGAATAAAGACGAGCCAGCACAGCCAGCTCATGCGTAACGGCTTTGTG
GAGCTGTCAAGGCCTGGTCTCTGGGAGAGAGGCACAGGGAGGCCAGACA
AGGAAGGGGTGACCTGGAGGGACAGATCCAGGGGCTAAAGTCCTGATAA
GGCAAGAGAGTGCCGGCCCCCTCTTGCCCTATCAGGACCTCCACTGCCA
CATAGAGGCCATGATTGACCCTTAGACAAAGGGCTGGTGTCCAATCCCA
GCCCCCAGCCCCAGAACTCCAGGGAATGAATGGGCAGAGAGCAGGAATG
TGGGACATCTGTGTTCAAGGGAAGGACTCCAGGAGTCTGCTGGGAATGA
GGCCTAGTAGGAAATGAGGTGGCCCTTGAGGGTACAGAACAGGTTCATT
CTTCGCCAAATTCCCAGCACCTTGCAGGCACTTACAGCTGAGTGAGATA
ATGCCTGGGTTATGAAATCAAAAAGTTGGAAAGCAGGTCAGAGGTCATC
TGGTACAGCCCTTCCTTCCCTTTTTTTTTTTTTTTTTTTTTTGTGAGAC
AAGGTCTCTCTCTGTTGCCCAGGCTGGAGTGGCGCAAACACAGCTCACT
GCAGCCTCAACCTACTGGGCTCAAGCAATCCTCCAGCCTCAGCCTCCCA
AAGTGCTGGGATTACAAGCATGAGCCACCCCACTCAGCCCTTTCCTTCC
TTTTTAATTGATGCATAATAATTGTAAGTATTCATCATGGTCCAACCAA
CCCTTTCTTGACCCACCTTCCTAGAGAGAGGGTCCTCTTGATTCAGCGG
TCAGGGCCCCAGACCCATGGTCTGGCTCCAGGTACCACCTGCCTCATGC
AGGAGTTGGCGTGCCCAGGAAGCTCTGCCTCTGGGCACAGTGACCTCAG
TGGGGTGAGGGGAGCTCTCCCCATAGCTGGGCTGCGGCCCAACCCCACC
CCCTCAGGCTATGCCAGGGGGTGTTGCCAGGGGCACCCGGGCATCGCCA
GTCTAGCCCACTCCTTCATAAAGCCCTCGCATCCCAGGAGCGAGCAGAG
CCAGAGCAT

Rat GFAP promoter [SEQ ID NO: 2]:

CCTGCAGGGCCCACTAGTCTGTAAGCTGGAAGTCTGGCAGTGCTGAGCT
GGCCAACCCCCTCAGGACCTCCTCCTTGTGCCCACTGAATGACTCACCT
TGGCATAGACATAATGGTCAGGGGCGGGCACACAGCCTGATTCCCGCTG
CACTCCAGGCCCCCTTCAATGCTTTCCGAGAAGTCCATTGAGCTGGGAG
CTTGTACTGCACCAAGGGCTGACATCCTGGCAGCCAGGGATGAAAGCAG
CCCATGGGGCTACCCTTGCCGTATGCCTCACTGGCGGCAGAGAACAAGG
CTCTATTCAGCAAATGCCCTGGAGTAGACACCAGAAGTCCAAGCATGGG
CAGAGGAAGGCAGGCGTTGGGGGCTGGAGGGGAGCAGAGCTGTCTGTTT
TCCAGAAGCCCAAGGGTACAGATGGCGCCTGGGGGGGAACTGAGTGGAG
GGGATAGATGGGCCTGAGATCTCAAACATCAACAGCCTCCTCCCCACCA
ACGATGAAGGTGGAGGTTGGTTTCCCAGACCTACATATCCCCCAGAGAC
CTGGTGTATGAAAATTCAAAGGAGGTAAGTCTCCTGAGAGAACGGGGGG
CTCACAAATGAAGCCAGCTGTCTTACCCTATCAGGACCTACGTGCATTC
CTTCTGTCCTGCCCCCTAAACACACAGCCAGAGGCTCAAATTGATTCTG
GAGTCACAAAGGGGGCTTGAAACCCCAGCCCCCCACTCCTGAACTCCAG
GAATGAGAAGATAGTATTGGAGGGGTTCAGAGGAGAGGGCTCTGCACAT
CTGTTGAGAATGGGGGTCCCAGGAGAGTGTAATTTAGGCTGATCCCGGA
GGAAGGGAATAGGCTCTTCAAGATCCTAGCATCTCACAGGCCCACAGAG
AAGTTCAGAGTTGGGGCAGCCCTGGCTTACAGGCTCTAAGAACTGGAGG
CAGTTTACCCAACCCAGCTGTGTGCATGCTGTCCCTCTCTCTGTCTCTG
TCTGTCTCTCTCTGTCTCTGTCTCTCTGTGTGTGTGTGTGTGTGCTCAC
ACACGTGTGTGTTTATCACACAAATGTTCATGTGTGTGTACATACATGT
GTTGAGGCCAGAGGTCAACCTCAGACACTGTTGACTTGGTTGTATGAGA
TAACATTTCCCCCTGGGACCTGGGATTTGCCAATTAGTGTGACCCAGGA
AGCCTACTTATTTTCATTCCTCAGCACTGCAGTTACAAGTATGCACTGT
CAAACCAGGCCTTTTTTTTTTTTTTTTTCCAAACCAGGCCTTTTGTATT
CGCTCTGTGGCTAGAACTTGGGTCTCCATGCTTGACAGGCAAGCGATTT
ATGGACTAAGCTGTTTCCTCGGCCCTCTCTTGACCCATTTACCAGAAAT
GGGGTTTCCTTGATCAATGGTTAAGCCAGGCTGGTGTTCCCAGGAAACC
CTTGACTCTGGGTACAGTGACCTTGGTGGGGTGAGAAGAGTTCTCTCCA
TAGCTGGGCTGGGGCCCAGCTCCACCCCCTCAGGCTATTCAATGGGGTG
CTGCCAGGAAGTCAGGGGCAGATCCAGTCCAGCCCGTCCCTCAATAAAG
GCCCTGACATCCCAGGAGCCAGCAGAAGCAGGGCAT

As used herein, “consists essentially of” or “consisting essentially of” means that the nucleic acid sequence includes one or more nucleotide bases, including within the sequence or at one or both ends of the sequence, but that the additional nucleotide bases do not materially affect the function of the nucleic acid sequence.

In addition to cell-type specificity, high-level expression of therapeutic genes is often desirable for cancer therapy. Compared to other cellular promoters, GFAP promoters possess a relatively strong activity, capable of driving expression of a transgene representing up to approximately 0.2% of total brain protein (Smith et al., Neurobiol Aging. (1998) September-October;19(5):407-13).

The nucleic acid molecule also contains a plurality of miRNA target sequences. An “miRNA target sequence” is a nucleic acid sequence that is recognized by, and is capable of binding an miRNA molecule, including an miRNA molecule incorporated in a RISC. Inclusion of an miRNA target sequence in the 3′ UTR of a gene may result in recognition and binding by a RISC containing the miRNA that binds to the target sequence, and may ultimately result in reduced expression of the gene, including reduced translation of the mRNA transcript of the gene.

An miRNA target sequence is capable of binding an miRNA, meaning that the target sequence is at least partly complementary to the miRNA sequence and has the capacity to be recognized by and may bind to the miRNA, depending on the concentration, the cellular context, the environmental conditions and the presence of other binding molecules. Similarly, reference to an miRNA target sequence as “for binding” an miRNA or that “binds” an miRNA means that the target sequence is capable of binding the miRNA but not that the target sequence is necessarily occupied at any given time by a bound miRNA or RISC.

Each miRNA target sequence contained within the plurality of miRNA sequences is selected based on relative levels of the miRNA in glioma cells as compared to normal glial cells. Target sequences are chosen for miRNA that are enriched in normal glial cells compared to glioma cells, and thus are relatively down-regulated in glioma cells in comparison to normal glial cells. For example, the miRNA level in a glial cell may be about 2 times or greater, about 3 times or greater, about 4 times or greater, about 5 times or greater, or about 10 times or greater, than in a glioma cell.

The level of an miRNA in a glial cell or glioma cell can be assessed using routine laboratory methods, including RNA quantification techniques, such as RNA microarray methods. Methods for assessing activity of miRNA in a glial cell or glioma cell may also be used, for example assaying for the ability of a given miRNA to silence a reporter gene that contains an appropriate miRNA target sequence. Examples of such techniques for assessing the level of an miRNA include those described in the following Example.

Target sequences for miRNA that are suitable for use in the nucleic acid molecule include the target sequences for has-miR-31, has-miR-127 or has-miR-143.

In particular embodiments, each miRNA target sequence comprises, consists essentially of or consists of the sequence of has-miR-31, has-miR-127 or has-miR-143, as shown below:

| | |
|---|---|
| Has-miR-31 [SEQ ID NO: 3]: | CAGCTATGCCAGCATCTTGCC |
| Has-miR-127 [SEQ ID NO: 4]: | AGCCAAGCTCAGACGGATCCGA |
| Has-miR-143 [SEQ ID NO: 5]: | TGAGCTACAGTGCTTCATCTCA |

Each miRNA target sequence within the plurality of miRNA target sequences may be the same or different as another miRNA sequence. In a certain embodiment, each miRNA target sequence is the same target sequence. In a different embodiment, each miRNA target sequence is a different target sequence. In a different embodiment, the plurality contains more than one different target sequence but at least two target of the individual miRNA target sequences are the same.

The nucleic acid molecule contains a plurality of miRNA target sequences. The number of individual miRNA target sequences within the plurality is a sufficient number to allow for down-regulation of expression of a transgene within normal a glial cell while allowing for expression of the transgene within a glioma cell. For example, the plurality of miRNA target sequences may be 2 or more, 3 or more, 4 or more, 5 or more, 6 or more, 7 or more, 8 or more, 9 or more or 10 or more individual miRNA target sequences. In certain embodiments, the plurality of miRNA target sequences contains 3 target sequences, 6 target sequences or 9 target sequences.

In particular embodiments, the plurality of miRNA target sequences contains one each of the target sequence for has-miR-31, has-miR-127 and has-miR-143, or contains 3 target sequences, all 3 being the target sequence for has-miR-31, has-miR-127 or has-miR-143. In other embodiments, the plurality of miRNA target sequences contains one each of the target sequence for has-miR-31, has-miR-127 and has-miR-143, repeated twice for a total of 6 target sequences, or contains one each of the target sequence for has-miR-31, has-miR-127 and has-miR-143, repeated three times for a total of 9 target sequences.

Without being limited to any particular theory, the level of repression seen with the miRNA may increase as the number of target sequences increase, suggesting that the number of miRNA target sites in an mRNA may influence the level of transgene repression, possible due to an increased probability of a single necessary binding event taking place. As well, repression of target mRNAs has been shown to be accomplished by the combinatorial action of different miRNA species (Bartel and Chen, 2004), and present results as set out in the Example below suggest that different miRNAs can act cooperatively to reduce transgene expression. Although only a handful of miRNA:mRNA interaction pairs are known in animals, there are already instances in which different miRNA species have been found to regulate the same targets (Bartel and Chen, 2004), for example, the miRNAs lin-4 and let-7 and their target mRNAs lin-14, lin-28, lin-41 and hbl-1 (Reinhart et al., 2000; Abrahante et al., 2003; Lin et al., 2003; Sempere et al., 2004). The silencing complexes may either mutually stabilize one another or cooperatively interact to more effectively inhibit transgene expression, or both mechanisms might be operative at the same time (Doench et al., 2003). These examples, as with other biological regulatory systems, most notably, cooperative interactions in gene regulation, would allow a cell to fine-tune the expression of an mRNA by regulating the degree of binding of different miRNAs to the 3′ UTRs of the mRNA (Bartel and Chen, 2004). This property of miRNA regulation may be beneficially used in the presently described nucleic acid molecule to enhance the suppression of transgene gene expression in normal glial cells.

The miRNA target sequences may be separated from each other by a linker sequence within the nucleic acid molecule. For example, 2, 4, 6, 8, 10 or more nucleotide bases may be inserted between each miRNA target sequence within the plurality of miRNA target sequences.

As stated above, the glial-specific promoter and plurality of miRNA target sequences are operably linked to a coding sequence of a transgene.

A first nucleic acid sequence is operably linked with a second nucleic acid sequence when the sequences are placed in a functional relationship. For example, a coding sequence is operably linked to a promoter if the promoter activates the transcription of the coding sequence. In a further example, a regulatory sequence such as an miRNA target sequence is operably linked to a coding sequence if that regulatory sequence is capable of regulating the expression of the coding sequence, including in cooperation with additional cis- or trans-acting factors, for example a RISC.

Thus, the transcription of the transgene is regulated at least in part by the glial-specific promoter and translation of the transgene is regulated at least in part by miRNA binding to the miRNA target sequences. This arrangement reduces expression of the transgene in cells that are not glial-derived, while the miRNA control reduces expression of the transgene in normal glial cells, which have a relative enrichment of relevant miRNAs.

The transgene may be any transgene that is desired to be expressed in a glioma cell, for example for diagnosis, detection, or treatment. The transgene may encode any expression product having clinical usefulness, such as a gene product or protein that is involved in glioma prevention or treatment, or a gene product or protein that has a cell regulatory effect that is involved in glioma prevention or treatment. The transgene product may be a protein or a peptide.

In certain embodiments, the transgene product directly targets the expression of a particular gene required for tumour cell growth or survival.

In certain embodiments, the transgene product includes a therapeutic protein or peptide. The therapeutic protein or peptide may substitute a defective or missing gene product, protein, or cell regulatory effect in the glioma cell, thereby enabling killing of a glioma cell or prevention or treatment of glioma cancer.

Therapeutic proteins and peptides include gene products that induce direct killing of the transfected or infected glioma cell (including HSV-tk, cytotoxins, tumour suppressor proteins such as p53, p51 or p71, apoptosis-inducers), immunomodulation proteins (such as IL-2, IL-4, IL-12, IFN and TNF-α), angiogenesis inhibitor proteins, tumour suppressor proteins, suicide proteins, apoptosis proteins, anti-angiogenic proteins and antibodies (including functional fragments of antibodies). The therapeutic protein may also be the bacterial DT-A protein.

In a particular embodiment, the therapeutic protein includes the p53 protein or a protein in the p53 apoptosis pathway (“p53 pathway protein”), which when expressed in a glioma cell may induced cell cycle arrest or apoptosis in the glioma cell. A p53 pathway protein is a protein located in the p53 apoptosis pathway that is involved in effecting or inducing apoptosis in a cell and may be upstream or downstream of p53 in the p53 apoptosis pathway. For example, the p53 pathway protein may be a protein that activates p53, that regulates p53 activity or expression, that is activated by p53 or that is expressed as a result of p53 activation, and that is involved in the apoptosis response related to p53 activation.

Over-expression of the wild type p53 gene is a strategy to either inhibit the growth of tumour cells or promote death of the cells by apoptosis, based on the observations that uncontrolled growth of many tumours results, at least in part, from the loss or mutation of the p53. gene. Restoring normal wild-type p53 function may also enhance apoptotic actions of radiotherapy and chemotherapy, when given in combination with such treatments. Several viral vectors have been tested for p53 gene delivery. One study reported the use of baculovirus vectors carrying the p53 gene under the control of the CMV promoter to kill Saos-2 tumour cells (Song et al., Exp Mol Med. (2001) March 31;33(1):46-53).

In another particular embodiment, the therapeutic protein is the DT-A protein. Under the control of a cell-type or tumour specific promoter, the DT-A gene has been tested in cancer therapy (Massuda, E. S., et al. Proc Natl Acad Sci USA (1997) 94:14701-6). This bacterial protein is highly toxic when introduced into the cytoplasm of eukaryotic cells and inhibits protein synthesis by catalyzing ADP ribosylation of the diphthamide group of cellular elongation factor 2 and kills cells through an apoptosis pathway (Michl, P. and Gress, T. M. Curr Cancer Drug Targets (2004) 4:689-702).

In another particular embodiment, the therapeutic protein is herpes simplex virus thymidine kinase (HSVtk). The thymidine kinase converts the non-toxic prodrug ganciclovir into a compound that is toxic to cells. Thus, expression of thymidine kinase, followed by ganciclovir treatment, is a current method employed in inducing tumour cell death.

The above-described nucleic acid molecule containing the glial-specific promoter and plurality of miRNA target sequences both operably linked to a coding sequence for a transgene can be synthesized using standard molecular biology and molecular cloning techniques known in the art, for example, as described in Sambrook et al. (2001) Molecular Cloning: a Laboratory Manual, 3^{rd} ed., Cold Spring Harbour Laboratory Press).

The nucleic acid molecule may be inserted into an expression vector for delivery to and stability in an expression system for use in an appropriate expression system that can support transcription from a glial-specific promoter. For example, the nucleic acid molecule may be included in a plasmid, chromosome, mitochondrial DNA, plastid DNA, virus, or nucleic acid fragment.

Thus, there is provided an expression vector comprising the present nucleic acid molecule. As stated above, the expression vector may be a plasmid, a chromosome including an artificial chromosome, a mitochondrial DNA, a plastid DNA, a virus, or a nucleic acid fragment.

In certain embodiments, the expression vector as provided is a viral vector. Baculovirus vectors are particularly suited for expression of a transgene in neuronal cells, or in glial cells, particularly astrocytes and cells derived from astrocytes.

Baculovirus infection does not lead to expression of its own genes or viral replication in mammalian cells (Carbonell et al., J Virol. (1985) October;56(1):153-60; Hofmann et al., Proc Natl Acad Sci USA (1995) 92: 10099-10103; Stanbridge et al., J Biomed Biotechnol. (2003) 2003(2):79-91). As a result, even though certain sequences of the virus could function as promoters or enhancer of transcription, they will be silent in mammalian cells due to the absence of supporting factors and less likely to influence the cell-type specificity of a mammalian promoter inserted into a baculovirus vector.

Thus, in one particular embodiment, the viral vector is baculovirus Autographa californica multiple nucleopolyhedrovirus (AcMNPV). AcMNPV-based vectors are emerging as a new generation of gene delivery vehicles (Hofmann et al., (1995), supra; Boyce et al., Proc Natl Acad Sci USA (1996) 93: 2348-2352; Sarkis et al., Proc. Natl. Acad. Sci. U.S.A. (2000) 97: 14638-43; Ghosh et al., Mol Ther (2002) 6:5-11; Kost and Condreay, Trends Biotechnol (2002) 20: 173-180; Kost T A, et al., Nat Biotechnol. (2005) 23:567-75).

Baculoviruses are unable to productively replicate and express viral proteins in mammalian cells, meaning these viruses can enter but not replicate in mammalian cells, thus significantly reducing the chance of pre-existing antiviral humoral and cellular immunity in mammals. Other intrinsic advantages that have made AcMNPV an attractive option as a gene delivery vector include a broad tropism for both proliferating and non-proliferating cells, the lack of obvious cytopathic effects, large cloning capacity and easy preparation of high titers of viruses.

Used as a gene vector for systemic delivery, baculoviruses are inactivated easily after exposed to serum complements (Hofmann et al., Gene Ther (1998) 5: 531-536). The central nervous system (CNS), protected by blood-brain barrier (BBB), is virtually isolated from circulating immunological factors including complement components (Carson and Sutcliffe, J Neurosci Res (1999) 55: 1-8), serving as a suitable organ for baculovirus-mediated gene expresMon. Direct injection of baculovirus vectors to the brain, using a thin needle and slow injection speed to avoid hemorrhage, usually gives satisfactory levels of transgene expression in the organ (Sarkis et al., (2000), supra; Li et al., Mol Ther. (2004) 10:1121-9; Li et al. Exp Physiol. (2005) 90:39-44). Attractively, baculoviruses display a high tropism for glial cells (Sarkis et al., (2000), supra). A previous study using Cy3-labled baculoviruses demonstrated that about 70% of virus-infected cells in the striatum were glial cells (Li et al., (2004), supra). Due to this factor in combination with the present nucleic acid molecule which incorporates a glial-specific promoter and relevant miRNA target sequences that are down-regulated in glioma cells relative to normal glial cells, expression of the transgene should be predominantly limited to glioma cells, thereby minimizing potential side effects on important functional healthy cells, of the central nervous system that could otherwise be elicited by expression of exogenous genes. Predominantly limited to glioma cells means that the expression is substantially lower in non-glioma cells, as described above.

A skilled person will be able to construct a suitable expression vector, including a viral vector and particularly a baculovirus vector, using known molecular biology and cloning techniques, and be able to test the ability of the constructed vector to deliver the nucleic acid molecule to a glioma cell. Similarly, a skilled person will be able to determine whether a particular viral vector influences the cellular specificity of expression from the present hybrid promoter using routine testing, for example, by monitoring expression of a reporter gene using various vector constructs in different cell types.

In particular, baculoviruses are well known and characterized and baculoviral vectors for mammalian gene transfection, including the use of Autographa Californica are known (see for example, Sarkis, C. et al. (2000) Proc Natl Acad Sci 97(26):14638-43). A skilled person can readily construct any suitable baculoviral vector for use in this invention. The baculovirus may be so modified using standard techniques that will be known to a skilled person, such as PCR and molecular cloning techniques. For example, baculovirus can be readily modified using commercially available cloning and expression systems such as the Bac-To-Bac™ Baculovirus Expression system (Gibco BRL, Life Technologies, USA).

The nucleic acid molecule, including when incorporated into a suitable expression vector, are useful for the expression of a transgene in a glioma cell, for example, an astrocytoma tumour cell or a tissue culture line derived from glioma or astrocytoma tumour cells.

Thus, there is presently provided a method for expressing a transgene in a glioma cell.

A glioma cell is transfected with the nucleic acid molecule, for example an expression vector comprising the above described nucleic acid molecule.

The method of transfection will depend on the nature of the cell that is to be transfected, the method of culturing or growing the cell that is to be transfected, as well as the particular expression vector that is to be used.

The glioma cell may be any glioma cell, including an astrocytoma, a neoplastic glial cell, a tumourous glial cell. The glioma cell may be an in vitro cell including an ex vivo cell explanted from a subject or may be a glioma cell in a subject.

Standard transfection methods for transfection of mammalian cells with nucleic acid molecules are known, and include transfection with carrier molecules such as liposomes, cationic polymers, cationic lipids and calcium phosphate, transfection by microinjection, needle-free injection, electroporation and using naked DNA.

Where the expression vector is a viral vector, the transfection may be achieved by exposing the cell to a virus particle containing the particular viral expression vector DNA under conditions which allow for infection of the cell by the virus, for example, by addition of live virus to culture medium or contacting the live virus with the outside of the cell.

Following transfection with the expression vector, the transfected cell is grown under conditions which allow for expression from the glial-specific promoter, including in the presence of any transcription factors or cell signals required for transcription from the particular promoter being used. For example, if the promoter is a hybrid promoter that requires a viral protein or a particular cellular protein not typically expressed in glial cells, such a growth factor or protein should be supplied in the growth medium or be expressed in the transfected cell, for example by inclusion of a gene encoding such a factor in the expression but under control of a promoter that does not require such a factor.

In another embodiment, there is provided a method for treating a glioma, which may be an astrocytoma, in a subject.

“Treating” refers to refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although, more preferably, it involves halting the progression of the disease permanently.

The method includes administering to a subject the above described nucleic acid molecule, for example inserted in an expression vector.

The subject is any subject in need of such treatment, including a mammal, particularly a human subject.

Methods for introducing the nucleic acid molecule into mammalian cells in vivo are known and may be used to administer the nucleic acid molecule of the invention, such as an expression vector, to a subject for treatment of glioma. A nucleic acid of the invention may be delivered to cells in a subject using methods such as direct injection of DNA, receptor-mediated DNA uptake, viral-mediated transfection or non-viral transfection and lipid based transfection, all of which may involve the use of expression vectors as described herein. A delivery apparatus (e.g., a “gene gun”) for injecting DNA into cells in vivo may be used. Such an apparatus may be commercially available (e.g., from BioRad).

To deliver the nucleic acid molecule specifically to glioma or astrocytoma cells in a particular region of the brain, the nucleic acid molecule, particularly a viral expression vector, may be administered by injection, including stereotaxic microinjection as is known in the art. For human patients, the stereotactic frame base will be fixed into the skull, and the brain with the stereotactic frame base will be imaged using high resolution MRI. Using appropriate stereotactic software, the images will be translated into 3 dimensional coordinates appropriate for the stereotactic frame for targeted injection of vector DNA.

The nucleic acid molecule may be administered in an amount sufficient to achieve the desired result, for example, expression of the transgene in the target cells. For example, the nucleic acid molecule may be administered in quantities and dosages necessary to express sufficient quantities of a therapeutic product which functions to alleviate, improve, mitigate, ameliorate, stabilize, inhibit, prevent including prevent the spread of, slow or delay the progression of, or cure a glioma.

The effective amount to be administered to a patient can vary depending on many factors such as the pharmacodynamic properties of the nucleic acid molecule, the mode of administration, the age, health and weight of the subject, the nature and extent of the disorder or disease state, the frequency of the treatment and the type of concurrent treatment, if any.

One of skill in the art can determine the appropriate amount based on the above factors. The nucleic acid molecule may be administered initially in a suitable amount that may be adjusted as required, depending on the clinical response of the patient. The effective amount of nucleic acid molecule can be determined empirically and depends on the maximal amount of the nucleic acid molecule that can be administered safely.

When the nucleic acid molecule comprises a viral expression vector, the virulence and titre of the virus will be a factor in determining the effective amount.

Particularly, when the nucleic acid molecule is in the form of a baculovirus, since baculovirus has little to no cytotoxicity in vertebrates, large doses may be tolerated. However, the amount of baculovirus administered should be the minimal amount that produces the desired result. In various embodiments, a dose of about 10⁹ plaque forming units (“pfu”) of baculovirus as described herein is administered to a human patient. In other embodiments, about 10² to about 10⁹ pfu, about 10⁶ to about 10⁹ pfu, about 10⁵ to about 10⁷ pfu, or about 10⁵ to about 10⁶ pfu may be administered in a single dose.

Effective amounts of baculovirus can be given repeatedly, depending upon the effect of the initial treatment regimen. Administrations are typically given periodically, while monitoring any response. It will be recognized by a skilled person that lower or higher dosages than those indicated above may be given, according to the administration schedules and routes selected.

The nucleic acid molecule may be administered as a sole therapy or may be administered in combination with other therapies, including chemotherapy, radiation therapy or other genetic treatments. For example, the nucleic acid molecule may be administered either prior to or following surgical removal of a primary tumour or prior to, concurrently with or following treatment such as administration of radiotherapy or conventional chemotherapeutic drugs. In one embodiment, the nucleic acid molecule can be administered in combination with, or in a sequential fashion with, an oncolytic virus that induces lysis of a cell that becomes infected with the oncolytic virus.

Also presently contemplated are uses of the various nucleic acid molecule constructs described herein for expressing transgene in a glioma, and for treating a glioma in a subject. Also contemplated are uses of the various nucleic acid molecule constructs described herein for preparation of a medicament for treating a glioma in a subject.

Transgenic cells and transgenic non-human animals comprising the present nucleic acid molecule or expression vector are also contemplated.

To aid in administration, the nucleic acid molecule or expression vector may be formulated as an ingredient in a pharmaceutical composition. The compositions may routinely contain pharmaceutically acceptable concentrations of salt, buffering agents, preservatives and various compatible carriers or diluents. For all forms of delivery, the nucleic acid molecule or expression vector may be formulated in a physiological salt solution.

The proportion and identity of the pharmaceutically acceptable diluent is determined by chosen route of administration, compatibility with a nucleic acid molecule, compatibility with a live virus when appropriate, and standard pharmaceutical practice. Generally, the pharmaceutical composition will be formulated with components that will not significantly impair the biological properties of the nucleic acid, particularly when it is a baculovirus expression vector. Suitable vehicles and diluents are described, for example, in Remington's Pharmaceutical Sciences (Remington's Pharmaceutical Sciences, Mack Publishing Company, Easton, Pa., USA 1985).

Solutions of the nucleic acid molecule may be prepared in a physiologically suitable buffer. Under ordinary conditions of storage and use, these preparations contain a preservative to prevent the growth of microorganisms, but that will not inactivate or degrade the nucleic acid molecule. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF 19) published in 1999.

The forms of the pharmaceutical composition suitable for injectable use include sterile aqueous solutions or dispersion and sterile powders for the extemporaneous preparation of sterile injectable solutions or dispersions, wherein the term sterile does not extend to any live virus that may comprise the nucleic acid molecule that is to be administered. In all cases the form must be sterile and must be fluid to the extent that easy syringability exists.

Kits and commercial packages containing the nucleic acid molecule constructs described herein, including an expression vector, or kits and commercial packages containing a pharmaceutical composition as described herein, are contemplated. Such a kit or commercial package will also contain instructions regarding use of the included nucleic acid molecule or pharmaceutical composition, for example, use to treat a glioma, or for expressing a transgene in a glioma cell.

The present nucleic acid molecules, expression vectors, methods and uses are further exemplified by way of the following non-limited example.

### EXAMPLE 1

To test the hypothesis that the use of a tissue-specific promoter could increase transgene expression preferentially to a specific type of tissues or cells while the additional use of miRNA regulation would provide another layer of control to differentiate transgene expression between tumour and normal cells of the same lineage, a baculoviral vector was constructed for glioma suicide gene expression in the brain.

The baculoviral vector harbours an engineered glial fibrillary acidic protein (GFAP) gene promoter to drive suicide gene expression in cells of glial origin while sparing functionally important neurons and other types of cells in the brain and in other organs in case of local or systemic leakage of intratumourally injected vectors.

In the current study, the GFAP promoter was used to target the expression of the HSVtk gene to cells of glial origin, thus avoiding any unintended deleterious effects on non-glial cells, either within or outside the central nervous system (CNS). However, the GFAP promoter, like many other tissue-specific promotes, has an expression profile that does not differ significantly between normal and tumour cells of the same lineage and HSVtk suicide gene expression followed by GCV treatment is toxic to normal astrocytes (Maron et al., 1997; Cowsill et al., 2000), necessitating the use of other control mechanisms to protect normal glial cells, which are abundant in the CNS and play a crucial role in supporting the survival and physiological functions of neurons.

Thus, the expression cassette of the vector also contains the target sequences of three miRNAs that are enriched in glial cells but down-regulated in glioma cells, in order to largely avoid side effects of the vectors that leak out of the tumour on nearby normal glial cells without interfering with suicide gene expression in tumour cells.

The combined use in a single expression vector of an engineered glial fibrillary acidic protein (GFAP) gene promoter and the repeated target sequences of three microRNAs that are enriched in glial cells but down-regulated in glioblastoma cells resulted in significantly improved in vivo selectivity of suicide gene expression. The incorporation of miRNA regulation into a transcriptional targeting vector adds an extra layer of security to prevent off-target transgene expression.

This Example set out below demonstrates that this dual control using transcriptional targeting and miRNA regulation resulted in significant reduction of non-target transgene expression in the brain.

Materials and Methods

Cell culture: Human cells used in this study were U87, H4, T98G, U118, U138, U251, A172, CCF, SW1783, and SW1088 glioblastoma cell lines, normal human astrocytes (NHA) and human embryonic stem cell-derived neurons. All glioblastoma cell lines were obtained from American Type Culture Collection (ATCC). U87, H4, T98G, U118, U138, U251, A172, and CCF cells were maintained in Dulbecco's modified Eagle's medium (DMEM) supplemented with 10% fetal bovine serum (FBS), 2 mM L-glutamine, 100 U/ml penicillin, 0.1 mg/ml streptomycin at 37° C., 5% CO₂ and SW1783 and SW1088 were cultured in Leibovitz's L-15 medium with fetal bovine serum (10%), 2 mM L-glutamine, 100 U/ml penicillin, 0.1 mg/ml streptomycin in a humidified incubator with 100% air. U87-luc cells that stably express luciferase were established as described before (Wang et al. 2006). Normal human astrocytes (NHA) from Clonetics Primary Cell Systems (Lonza, Basel, Switzerland) were cultured in special Astrocyte Basal Medium (ABM) supplemented with the AGM SingleQuots (Lonza) supplemented with 10 μg/ml human epidermal growth factor, 10 mg/ml insulin, 25 μg/ml progesterone, 50 mg/ml transferrin, 50 mg/ml gentamicin, 50 μmg/ml amphotericin, and 10% FBS. These primary astrocyte cultures were established from normal human brain tissue and are cryopreserved after secondary or tertiary passage. Human embryonic stem-derived neurons were generated based on a protocol described by Reubinoff et al (Reubinoff et al. 2001).

miRNA expression analysis: For miRNA analysis, small RNA was isolated from glioblastoma cell lines and normal human astrocytes using PureLink miRNA Isolation Kit (Invitrogen) in accordance with manufacturer's instruction. Small RNA samples were quantified spectrophotometrically and their purity was determined using RNA 6000 NanoChip on the Agilent 2100 Bioanalyzer (Agilent Technologies, Inc. CA, USA). The NCode Multi-Species miRNA Microarray Version 2 (Invitrogen) was used for high-throughput analysis. The array consists of optimized probe sequences for all mature miRNAs catalogued in miRBase Sequence Database, Release 9.0 (Griffiths-Jones et al. 2006) printed on epoxide-coated glass slides. There are 553 triplicate probes corresponding to human miRNAs on the array. A dual-colour labeling system was adopted to compare expression level of miRNAs in glioblastoma cells to NHA levels. Small RNA (500 ng) from NHA were sequence-tagged with AlexaFluor3 ligation mix after poly (A)-tailing as per the NCode miRNA Labeling System (Invitrogen). Similarly, but independently, equivalent amount of small RNA from glioma cell lines were tagged with AlexaFluor5 ligation mix. The ligation mix tags a capture sequence to the small RNA, providing specificity for the subsequent binding of either AlexaFluor3 capture reagent or AlexaFluor5 capture reagent to the hybridized miRNA. Tagged NHA and glioma samples were co-hybridised overnight on triplicate arrays for each glioma cell line. After this initial hybridization, a mixture of AlexaFluor3- and AlexaFluor5-labeled 3DNA dendrimers (Genisphere) was applied to each microarray for detection and a second hybridization was performed for 4 hours. The dendrimers carry ˜900 AlexaFluor fluorophores, as well as specific sequences complementary to the ligated tags on the hybridized miRNAs and allow the detection of short hybridised targets. All intermediate and the final washing steps were performed as recommended, followed by scanning the slides on GenePix 4000B scanner (Molecular Devices Corporation, Sunnyvale, Calif., USA) at 532 and 635 nm for AlexaFluor3 (green) and AlexaFluor5 (red) signals respectively. All hybridizations were performed at the Microarray Centre of Biopolis Shared Facilities (A*Star, Singapore) using the MAUI Hybridisation system (Biomicro Systems, Salt Lake City, Utah) with MAUI Mixer hybridization chambers.

Intensity data was acquired using GenePixPro 6.0 software (Molecular Devices) after visually flagging absent probe spots or artefactual hybridizations and manually adjusting grid to align spots on the scanned slide images. The resultant *.GPR (GenePix Results) files were imported to GeneSpring GX 7.3 (Silicon Genetics, Redwood City, Calif.) for further analysis. Each probe spot contained intensity value for NHA (green/control) and for glioma (red/raw). Median pixel intensity values were background-subtracted and triplicate spots on each slide were automatically processed to their average by GeneSpring for both channels. Using normalization options on GeneSpring, per-spot normalization was done by dividing each spot's intensity by its control channel value for each slide; if control channel was below 10 then 10 was used instead. Per-slide normalization was done to the 50^{th} percentile of measurements on particular slide. Biological triplicate slides for each combination of gliomblastom cell line vs. NHA were grouped and averaged to represent a condition, generating ten conditions having intensity ratios calculated as glioma (red) signal vs. NHA (green) signal. To obtain reliable data only for miRNA probes that are considered present, each data file under consideration was filtered on ‘Flag filter’ to exclude absent probe spots. To identify differentially expressed . miRNAs in gliomblastom cell lines, a fold-change cut-off of 2×, (intensity ratio is <0.5 or >2), was used and further stringency filter for statistical significance (t-test p-value<0.05) was applied.

Real-time PCR of microRNAs was performed based on a reported protocol (Shi and Chiang, 2005) with slight modifications. Total RNA was isolated from three glioblastoma cell lines (U87, H4, T98G) and NHA using TRIzol reagent (Invitrogen, Singapore) and treated with TURBO DNA-free™ DNase (Ambion). Individually, the DNAse-treated total RNA (1 μtg) was polyadenylated with ATP by poly(A) polymerase using Poly(A) Tailing Kit (Ambion) according to the manufacturer's instructions. Polyadenylated total RNA was purified using RNeasy Mini Kit (Qiagen) using an adapted protocol. Briefly, 100% ethanol was added to the mixture of polyadenylated RNA and lysis buffer to a final ethanol concentration of 70% v/v which allowed, for the binding of the small RNA fraction to the column. The purified polyadenylated RNA was reverse transcribed with 200 U Superscript™ III Reverse Transcriptase (Invitrogen) and 0.5 μg poly (T) adapter [3′ rapid amplification of complementary DNA ends (RACE) adapter in the FirstChoice® RLM-RACE kit; Ambion] according to the manufacturer's protocol for RT-PCR (Invitrogen). The forward primer for real-time PCR analysis was designed based on entire known mature miRNA sequence, with additional 3 ‘A’s at the 3′ end improve amplification specificity. An exception was made for hsa-miR-143 primer which has 2 bases truncated at the 5′ end due to complementarity between the first and last 4 bases of the mature miRNA sequence. 5S rRNA was selected as the internal reference gene for PCR quantification. The sequence of the primers used are as follow: hsa-miR-31 (5′-GGCAAGAUGCUGGCAUAGCUGAAA) [SEQ ID NO.: 6], hsa-miR-127 (5′-UCGGAUCCGUCUGAGCUUGGCUAAA) [SEQ ID NO.: 7], hsa-miR-143 (5′-AGAUGAAGCACUGUAGCUCAAA) [SEQ ID NO.: 8], 5S (5′-CCGCCTGGGAATACCGGGTGCTGTAGGCTTT) [SEQ ID NO.: 9]. The reverse primer used was a 3′ adapter primer (3′ RACE outer primer in the FirstChoice® RLM-RACE kit).To determine absolute copy number, a standard curve was generated using a synthetic miR-26a RNA oligonucleotide (1^{st} Base, Singapore). Assuming that each cell contains 30 pg of total RNA, C_{T} values for each sample reaction were converted to absolute copy number based on this curve. All primers used were synthesized by Sigma-Proligo Singapore Pte Ltd (Biopolis Way, Singapore). Primer optimization was performed according to the protocol for Two-Step RT-PCR Master Mix for Primer Optimization (Applied Biosystems). Real-time PCR was performed on Rotor-Gene 6000 (Corbett Life Science) with the following conditions: 95° C. for 10 min followed by 45 cycles of amplification (95° C. for 15 s, 60° C. for 1 min). All reactions were run in triplicate. PCR specificity was checked by melting curve analysis.

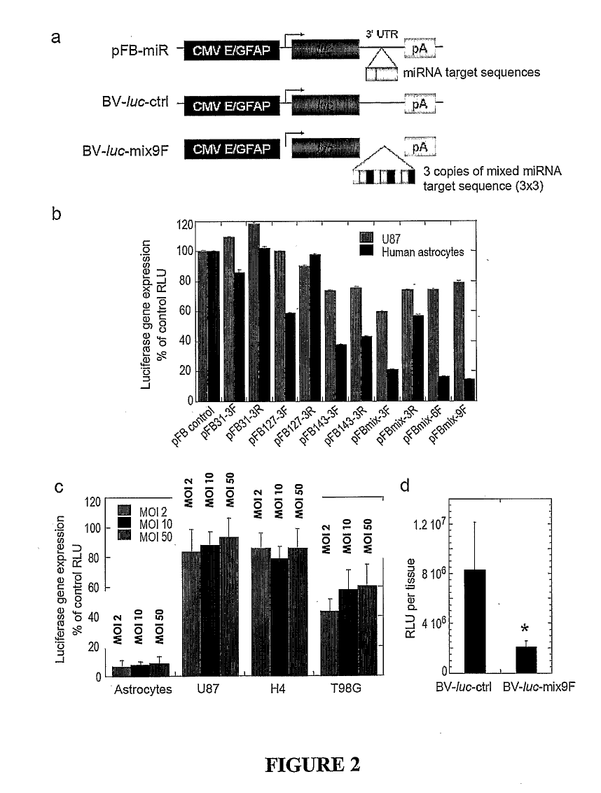

Construction of gene transfer vectors: To construct plasmid vectors, a recombinant pFastBac1 vector, pFB-CMV E/GFAP-luc previously generated and containing an engineered cell-type specific promoter (Wang et al., 2006), was used as a starting backbone. This construct contains the firefly luciferase (luc) gene under the control of the promoter of glial fibrillary acidic protein (GFAP) encoding gene, the activity of which is augmented by placing the enhancer of human cytomegalovirus immediate early gene upstream of the GFAP promoter (CMV E, Wang and Wang, 2006). The HindIII site right after the luc gene in the pFB-CMV E/GFAP-luc vector was used to insert miRNA target sequences. The mature miRNA sequences of selected miRNAs were obtained from the miRNA registry (Griffiths-Jones et al., 2006). Four types of oligonucleotides were designed as 3X-mir-31, 3X-miR-127, 3X-miR-143, and Mix-mir. The first three contains three tandem copies of a sequence (in lower-case letters) designed to be perfectly complementary to the respective miRNA while the Mix-mir construct contains one copy each of the three different miRNA complementary sequence in the order given (Table 1 and FIG. 2a). Each miRNA target sequence is separated by a four nucleotide linker. The oligonucleotides were first phosphorylated at the 5′ ends using T4 Polynucleotide Kinase (New England Biolabs, Beverly, Mass., USA). The respective phosphorylated top and bottom strands of the oligonucleotides were annealed and subclone into the HindIII site. Constructs containing varying copies of the insert (miRNA complementary sequence) in both the forward and reverse orientations were selected after sequencing.

To construct baculoviral vectors, the Bac-to-Bac baculovirus expression system (Invitrogen) was used. Two luciferase containing pFB constructs (pFB-CMV E/GFAP-luc and pFBmix-9F) were selected for bacmid production. To generate baculoviral vectors with a herpes simplex virus thymidine kinase (HSVtk) gene, the luciferase gene in pFB-CMV E/GFAP-luc and pFBmix-9F vector was replaced with a HSVtk gene from pORF-HSVtk (InvivoGen, San Diego, Calif., USA). Recombinant baculoviruses were produced and propagated in Spodoptera frugiperda (Sf9) insect cells pre-adapted to Sf-900 II serum-free medium (Invitrogen). Sf9 cells were grown in spinner flasks at 27.5° C. Budded viruses in the insect cell culture medium were collected and filtered through a 0.45-μm pore size filter (Millipore, Bedford, Mass., USA) to remove any contamination, and concentrated by ultracentrifugation at 28,000 g for 60 min. Viral pellets were re-suspended in appropriate volumes of 0.1 M phosphate-buffered saline (PBS) and their infectious titers (plaque-forming units, pfu) were determined by plaque assay on Sf9 cells.

In vitro transfection, transduction and transgene expression analysis: Cells were seeded separately in 48-well plate at a density of 20,000 per well one day before transfection. Transfection of the different constructs and the control plasmid without any miRNA target sequence (pFB-CMV E/GFAP-Luc) was performed with Lipofectamine 2000 (Invitrogen). For each transfection reaction, 0.2 μg of plasmid DNA and 0.5 μL of Lipofectamine diluted in OPTI-MEM I Reduced Serum Medium were added to cells and incubated for 4 hours at 37° C., 5% CO₂. After the incubation, OPTI-MEM I containing the transfection complexes was replaced by normal growth medium and further cultured at 37° C. for 1 day. For baculoviral transduction, cells were incubated with baculoviruses for 1 hr, followed by removal of viruses and addition of normal growth medium. Cells were further cultured at 37° C. for 1 day before used for transgene expression analysis.

For luciferase activity assay, the transfected cells were washed once with PBS and freeze-thawed with 100 μL of reporter cell lysis buffer (Promega, Wis., USA). Ten μl of the cell extract was used for luciferase assay with an assay kit from Promega. Measurements were made in a single-tube luminometer (Berthold Lumat LB 9507, Bad Wildbad, Germany). The relative light units (RLU) were normalized to the protein concentration measured with Bio-Rad protein assay kit. HSVtk gene expression was examined using Western blot analysis. Cells were collected and lysed 24 hrs after viral transduction. Lysates with 5 μg of total protein were loaded into a 4-12% NuPAGE gradient gel (Invitrogen) for protein separation. After transferring proteins on nitrocellulose membrane, protein detection was performed using chemiluminiscence (ECL) detection system (Amersham Pharmacia Biotech, Piscataway, N.J., USA). The primary antibody used was a rabbit polyclonal antibody for HSVtk (kindly provided by Dr William Summers, Yale University, New Haven, Conn.), used at a dilution of 1:1000. Secondary anti-rabbit IgG (Sigma-Aldrich, St. Louis, Mo., USA) was used at a dilution of 1:3000. β-tubulin was used as a loading control and was detected using mouse monoclonal antibody against β-tubulin (Sigma) at a dilution of 1:5000.

Cytotoxicity assay: Cells were seeded in 48-well plates at a density of 10,000 cells per well. After allowing cells to grow overnight, the medium was changed to fresh growth medium containing various concentrations of ganciclovir (GCV) (InvivoGen). After one day incubation at 37° C., cells were infected with 50 multiplicity of infection (MOI) of baculoviral vectors (BV-HSVtk-ctrl and BV-HSVtk-mix9F). The medium was changed every two days after initial seeding to fresh growth medium containing appropriate amounts of GCV. Six days after virus infection, each well was replaced with fresh growth medium without GCV and 50 μl of a proliferation assay solution (CellTiter 96 Aqueous One Solution Cell Proliferation Assay, Promega) was added to each well, followed by 4 hours of incubation at 37° C. in a humidified incubator with 5% CO₂. The plate was transferred to a microplate reader (Bio-Rad, Hercules, Calif., USA) to measure the absorbance at a wavelength of 490 nm. Triplicate determinations for each treatment were performed. The relative cell growth (%) related to control cells (infected cells without GCV treatment) was calculated by (absorbance of sample−absorbance of blank)/(absorbance of control−absorbance of blank)×100%.

Animal experiments: Adult female Balb/c nude athymic immuno-incompetent nude mice (weighing 20 g, 6-8 wks old) were used. Animals were anesthetized by 150 mg/kg Ketamine and 10 mg/kg Xylazine and positioned in a stereotaxic instrument with the nose bar set at 0. Three μl of baculovirus (10⁷ pfu) in PBS was injected into either side of the striatum of the mouse brain (anteriorposterior: 0.0 mm, mediolateral: +2.0 mm and dorsoventral: −3.0 mm from bregma and dura) using a 100 Hamilton syringe connected with a 30 G needle at a speed of 0.6 μl/min. The needle was withdrawn slowly at the end of injection after 5 minutes.

For experiments investigating effects of HSV-TK/GCV treatment on the normal brain, brain injection of baculoviral vector was followed by daily intraperitoneal injection of either GCV at a dose of 50 mg/kg or PBS for 7 days. Mice were euthanized with an overdose of ketamine/xylazine, cardiac perfusion was performed using PBS and the brain tissues were collected for western blot analysis using a primary antibody against GFAP (Chemicon). For immunocytochemical staining, cardiac perfusion of PBS was followed by freshly prepared cold paraformaldehyde (4% in PBS). Brain specimens were left in cold 4% paraformaldehyde from 4 hours to overnight, cryo-protected by incubation in 30% sucrose till the specimens sank, and sectioned at −20° C. in 25 μm sections after being frozen in embedding medium (Jung tissue freezing medium, Leica Instruments, Germany). Sections were stained using a free-floating method. Sections were incubated in 0.3% Triton-X and 3% rabbit serum in PBS containing the primary antibody against GFAP at 1:100 dilution and 4° C. overnight. This was followed by three PBS wash steps and subsequent incubation with FITC-conjugated anti-mouse immunoglobulin secondary antibody (Sigma) at 1:100 dilution for 2 hours at room temperature. After PBS wash, brain sections were mounted with mounting medium (Vectashield). Images were viewed and saved under Olympus microscope.

For experiments investigating therapeutic effects, mice were inoculated with 0.5×10⁶ of human glioma U87-luciferase (U87-luc) cells into both sides of the striatum first. One week later, baculoviral vectors were injected into the tumours, followed by daily intraperitoneal injection of GCV. Tumour growth in the mouse brains were monitored by bioluminescent imaging of U87-luc cells in isoflurane gas-anaesthesized animals. Animals were injected intraperitoneally with D-luciferin (Promega) at 100 mg/kg in PBS just before imaging. Bioluminescent imaging was then performed with the IVIS imaging system coupled with cool CCD camera (Xenogen, Alameda, Calif., USA) at 10 and 20 min after Luciferin injection. Images acquisition and bioluminescent signals were measured by subtracting the background signal from the ROI signal and the photon intensity was analyzed with the Xenogen living imaging software v2.5.

In the handling and care of animals, the Guidelines on the Care and Use of Animals for Scientific Purposes issued by National Advisory Committee for Laboratory Animal Research, Singapore was followed. The experimental protocols of the current study were approved by the Institutional Animal Care and Use Committee (IACUC), National University of Singapore and Biological Resource Center, the Agency for Science, Technology and Research (A*STAR), Singapore.

Results

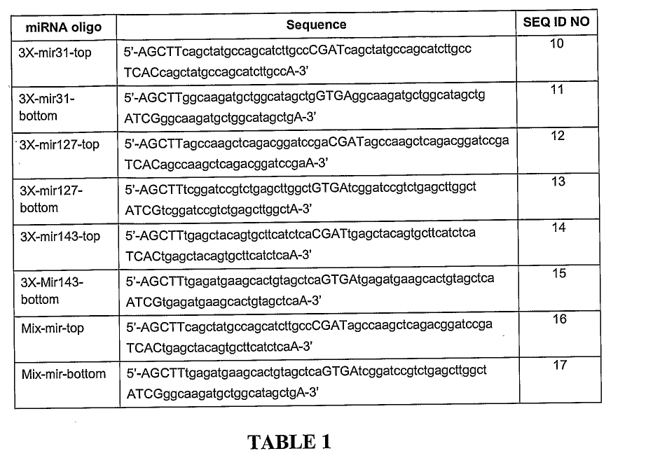

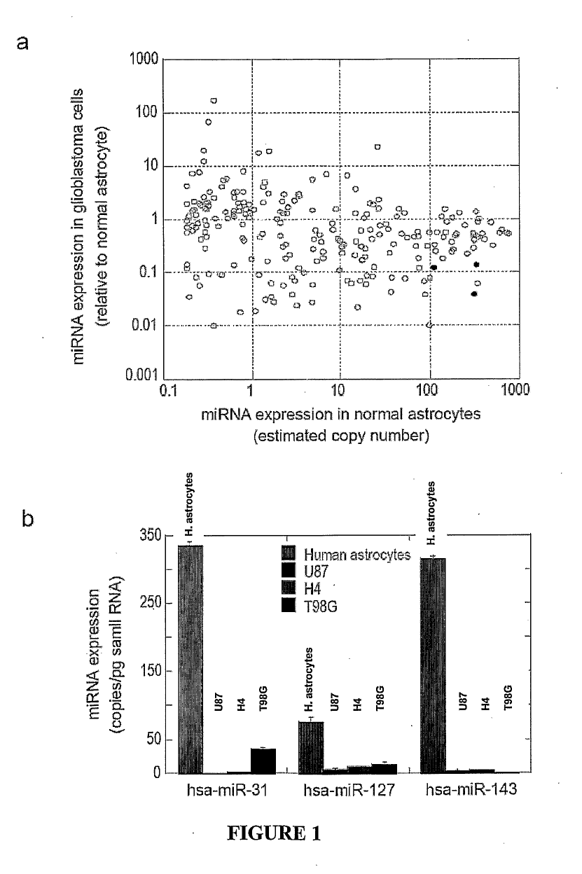

Profiling endogenous miRNA expression is important for predicting miRNA activity and enabling rational design of vectors that are subjective to miRNA regulation. Using miRNA microarray chips capable of examining 553 human miRNAs simultaneously, global expression changes of miRNAs were evaluated in 10 human glioma cell lines versus normal human astrocytes. miRNAs expressed in all samples with a tumour sample/normal sample ratio≧2 were considered as up-regulated miRNAs and the ratio≦0.5 as down-regulated miRNAs. 57 up-regulated and 129 down-regulated miRNAs were identified. The copy number of different miRNAs in normal astrocytes was estimated based on the average signal intensity of 10 microarray assays and the absolute copy number of miR-31 quantified using real-time PCR. Using significant downregulation in glioma cells and high copy number in normal astrocytes as the criteria, as well as taking into consideration the published miRNA expression levels in primary glioma samples and in the normal brain (Ciafre et al., 2005; Landgraf et al., 2007), three miRNAs were selected to design gene transfer vectors with a dual control system: has-miR-31, has-miR-127 and has-miR-143 (FIG. 1a). Quantitative real-time PCR analysis confirmed down-regulation of the 3 miRNAs in glioma cell lines and their relatively high levels of expression in normal astrocytes (FIG. 1b).

To construct dual control vectors, an engineered GFAP promoter (Wang et al, 2006) was used to restrict transgene expression in glial cell lineage and introduced miRNA target sequences with perfect complementarity to miR-31, miR-127 or miR-143 into the 3′-untranslated region of a transgene gene to restrict transgene expression in glioma cells (FIG. 2a). Three consecutive repeats of each miRNA target sequence (resulting in 3 copies of a particular miRNA target sequence), or 1, 2 or 3 sets of consecutive sequences of the three miRNA target sequences (resulting in mixed 3, 6 and 9 copies of the different types of target sequences) were inserted into each vector and the copies of target sequences were separated by a four nucleotide linker (Table 1). Since only one restriction enzyme site (HindIII) was used for insertion, the constructs were selected with either forward or reverse orientation.

After including the above regulatory elements into plasmid expression vectors with a firefly luciferase reporter gene, the ability of miRNA target sequences to repress luciferase gene expression after transfection in normal human astrocytes and U87 glioma cells were tested (FIG. 2b). In tumour cells, levels of inhibition were usually less than 20%, expect a 40% inhibition for a construct with mixed 3 target sequences and forward orientation. In normal astrocytes, the levels of inhibition were also low for the constructs containing 3 tandem copies of one type of target sequences. However, much greater levels of inhibition were achieved by using the constructs bearing a mixed combination of the three miRNA binding sites, especially when the number of binding sites increased to a total of 6 or 9 target sequences. Thus, pFBmix-6F and pFBmix-9F, which have 2 and 3 copies of each of the target sequence for the three selected miRNA respectively, displayed 80% inhibition of transgene expression in normal human astrocytes.

Insect baculoviruses were demonstrated previously to be efficient vectors, able to transduce astrocytes and gliomblastoma cells and to suppress in vivo glioma growth by over-expression of a toxic suicide gene (Wang et al., 2006). In this study, the funcitionality of miRNA regulation in baculoviral vectors was tested. Since the combination of three types of miRNA target sequences with a total of 9 copies (mix9F) is the most efficient construct in repressing transgene expression, it was used in baculoviral vectors to suppress transgene expression in subsequent experiments. The baculoviral vector with a luciferase gene and the miRNA target sequence was designated as BV-luc-mix9F and the control baculoviral vector as BV-luc-ctrl (FIG. 2a). When comparing the transduction efficiencies of the two vectors in glioma cell lines, slight-to-moderate reduction was observed with incorporation of the miRNA target sequence, from 7% in U87 cells, 14% in H4 cells to 40% in T98G cells (FIG. 2c). The different levels of inhibition of transgene gene expression correlated to the relative abundance of the three miRNAs in the three glioma cell lines (FIG. 1b), with U87 cells that express the lowest levels of the three miRNAs being less affected. However, in normal human astrocytes that express high levels of the three miRNAs, a 91% reduction in gene expression level was observed (FIG. 2c). To investigate in vivo inhibition by baculoviral vectors, the transduction efficiencies of the above two viral vectors in the brain of mice was compared, which, owing to miRNA sequence conservation between human and mouse, express the exact homologs of the three human miRNAs that were tested in the in vitro experiments. Two days after injection of the viral vectors into the striatum of nude mice, a lower level of luciferase expression from BV-luc-mix9F was observed. Although the in vivo suppressing effect of the miRNA target sequence was more moderate than in vitro, the inhibition was still about 4-fold (FIG. 2d).

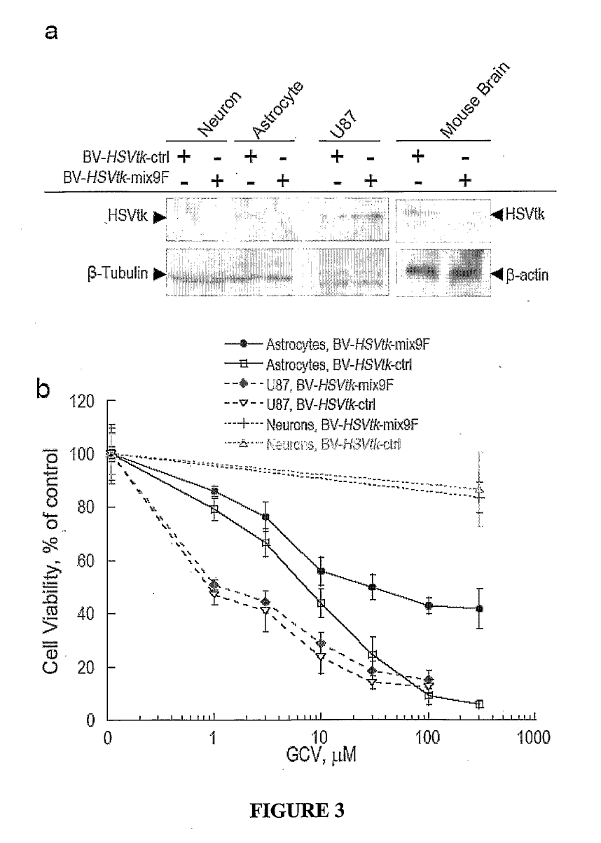

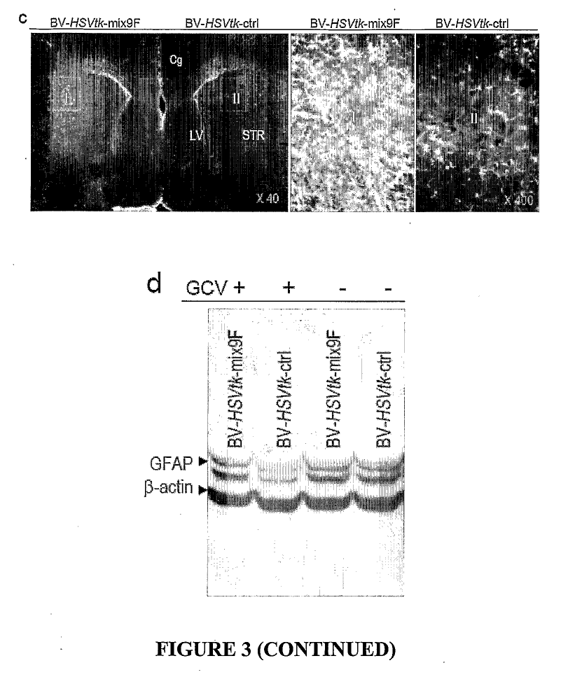

To test whether the mix9F miRNA target sequence is able to suppress the expression of a functional gene, two baculoviral vectors containing the herpes simplex virus thymidine kinase (HSVtk) suicide gene under the control of the engineered GFAP promoter were constructed, one with the mix9F target sequence (BV-HSVtk-mix9F) and one without the target sequence (BV-HSVtk-ctrl). Using Western blot analysis, HSVtk expression was detected in human U87 glioblastoma cells, with no difference in expression level after transduction using BV-HSVtk-ctrl and BV-HSVtk-mix9F (FIG. 3a). In normal human astrocytes and the mouse striatum, HSVtk expression was observed after BV-HSVtk-ctrl transduction, but not after BV-HSVtk-mix9F transduction (FIG. 3a). There was no detectable HSVtk expression in neurons derived from human embryonic stem cells after either HSVtk-ctrl or BV-HSVtk-mix9F transduction (FIG. 3a). in vitro effects of HSVtk expression followed by ganciclovir (GCV) treatment was further investigated and a GCV dose-dependent decrease in cell viability was found in both normal human astrocytes and U87 cells, but not obviously in human neurons (FIG. 3b). In U87 cells, there was no significant difference in cytotoxic potency between BV-HSVtk-ctrl and BV-HSVtk-mix9F, suggesting that incorporation of the mix9F miRNA target sequence does not interfere with HSVtk-related cell killing effects in glioma cells. In normal human astrocytes, the killing curves from the two baculoviral vectors were very different, with BV-HSVtk-ctrl inducing much more pronounced cell death than BV-HSVtk-mix9F at GCV concentrations of 10 μM and above. At the highest dose tested (300 μM), BV-HSVtk-ctrl induced death in more than 90% cells, whereas BV-HSVtk-mix9F induced ˜55% cell death. The IC₅₀ values obtained by GCV treatment (concentration of GCV that inhibited cell survival by 50%) were 0.8 μM and 1 μM in U87 glioblastoma cells and 7 μM and 30 μM in normal astrocytes after transduction with BV-HSVtk-ctrl and BV-HSVtk-mix9F respectively, indicating a 4-fold improvement of resistance to HSVtk/GCV-mediated cytotoxicity in non-target normal astrocytes. The different cell killing efficiencies by BV-HSVtk-ctrl in U87 cells and normal astrocytes indicate that the inhibition of DNA synthesis by GCV is more toxic for actively proliferating tumour cells. The difference by BV-HSVtk-mix9F between U87 tumour cells and normal astrocytes is at least partially attributed to the presence of the mix9F target sequence as well as the different susceptibilities of the two types of cells to GCV. Together, these results demonstrate that glioma cells and normal astrocytes display different vulnerabilities to HSVtk/GCV-mediated cellular toxicity, including in particular when BV-HSVtk-mix9F is used.

To examine whether the mix9F miRNA target sequence provides in vivo protection against HSVtk/GCV-induced cytotoxicity in the brain, BV-HSVtk-ctrl or BV-HSVtk-mix9F was injected into the striatum of nude mice followed by daily intraperitoneal injection of GCV for 7 days. Immunostaining of the brain sections demonstrated a significant decrease in the number of GFAP-positive cells in the striatum injected with BV-HSVtk-ctrl, but numerous cells strongly stained with the antibodies against GFAP on the contralateral side of the same brain injected with BV-HSVtk-mix9F (FIG. 3c). Similarly, Western blot analysis of the striatum tissues revealed noticeable decrease in the staining intensity of GFAP bands in the animals injected with BV-HSVtk-ctrl/GCV and no obvious change in the GFAP bands in the animals injected with BV-HSVtk-mix9F/GCV (FIG. 3d). Theses results indicate that mix9F-mediated suppression is efficient in protecting normal astrocytes in the brain.

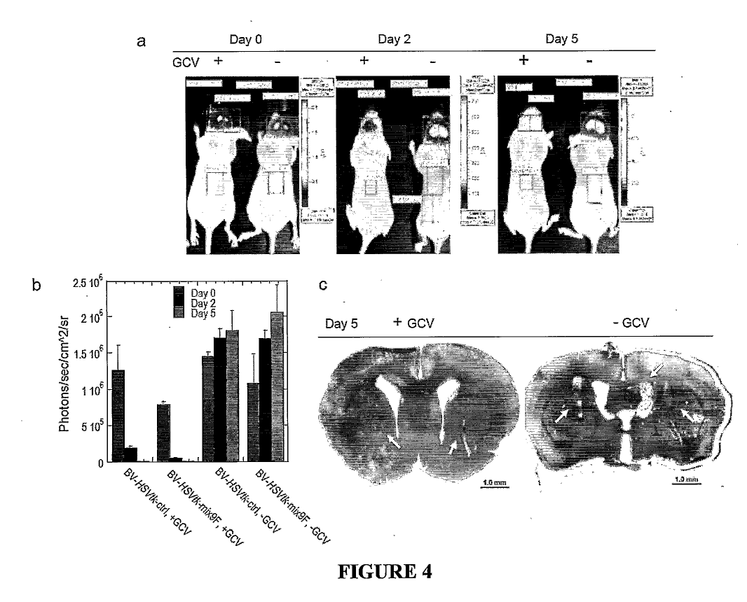

To investigate whether baculoviral vectors with a miRNA targeting sequence retained in vivo anti-tumour potency, U87-luc cells, a modified human glioblastoma cell line that stably expresses luciferase, were inoculated into the striatum of nude mice, followed by a single injection of baculoviral vectors into U87 glioblastoma xenografts 7 days later and daily intraperitoneal injection of GCV afterwards for 5 days. Tumour growth in the brain of living mice was monitored using a noninvasive bioluminescent imaging method. FIG. 4a shows the easily detectable luminescent activity from the inoculated U87-luc cells before virus injection and continuous increase in luminescent intensity during the 5-day observation period in an animal injected with BV-HSVtk-ctrl and BV-HSVtk-mix9F into the tumour xenografts, but without GCV injection. In an animal received GCV injection after intratumour injection of the baculoviral vectors, there was almost no detectable luciferase activity at day 5 post-injection of the viral vectors. Quantitative results from a group of 4 mice for each treatment are summarized in FIG. 4b, showing an obvious tumour eradication by a single injection of either BV-HSVtk-ctrl or BV-HSVtk-mix9F followed by GCV injection for 5 days, with no significant difference in the tumour suppressing effect between the two viral vectors. Tumour growth in the control animals and inhibition in the animals received virus and GCV injection was confirmed by histological examination (FIG. 4c).

### REFERENCES

1 Abrahante J E et al. The Caenorhabditis elegans hunchback-like gene lin-57/hbl-1 controls developmental time and is regulated by microRNAs. Dev Cell 2003; 4: 625-637.

2 Babak T et al. Probing microRNAs with microarrays: tissue specificity and functional inference. Rna 2004; 10: 1813-1819.

3 Bartel D P, Chen C Z. Micromanagers of gene expression: the potentially widespread influence of metazoan microRNAs. Nat Rev Genet 2004; 5: 396-400.

4 Birney E et al. Identification and analysis of functional elements in 1% of the human genome by the ENCODE pilot project. Nature 2007; 447: 799-816.

5 Brown B D et al. Endogenous microRNA can be broadly exploited to regulate transgene expression according to tissue, lineage and differentiation state. Nat Biotechnol 2007; 25: 1457-1467.

6 Brown B D et al. Endogenous microRNA regulation suppresses transgene expression in hematopoietic lineages and enables stable gene transfer. Nat Med 2006; 12: 585-591.

7 Bushati N, Cohen S M. microRNA functions. Annu Rev Cell Dev Biol 2007; 23: 175-205.

8 Chen J et al. A glial-specific, repressible, adenovirus vector for brain tumour gene therapy. Cancer Res 1998; 58: 3504-3507.

9 Ciafre S A et al. Extensive modulation of a set of microRNAs in primary glioblastoma. Biochem Biophys Res Commun 2005; 334: 1351-1358.

10 Condorelli D F et al. Tissue-specific DNA methylation patterns of the rat glial fibrillary acidic protein gene. J Neurosci Res 1994; 39: 694-707.

11 Cowsill C et al. Central nervous system toxicity of two adenoviral vectors encoding variants of the herpes simplex virus type 1 thymidine kinase: reduced cytotoxicity of a truncated HSV1-TK. Gene Ther 2000; 7: 679-685.

12 Doench J G, Petersen C P, Sharp P A. siRNAs can function as miRNAs. Genes Dev 2003; 17: 438-442.

13 Eng L F. Glial fibrillary acidic protein (GFAP): the major protein of glial intermediate filaments in differentiated astrocytes. J Neuroimmunol 1985; 8: 203-214.

14 Esquela-Kerscher A, Slack F J. Oncomirs—microRNAs with a role in cancer. Nat Rev Cancer 2006; 6: 259-269.

15 Filipowicz W, Bhattacharyya S N, Sonenberg N. Mechanisms of post-transcriptional regulation by microRNAs: are the answers in sight? Nat Rev Genet 2008; 9: 102-114.

16 Fukuyama K et al. Analysis of glial fibrillary acidic protein gene methylation in human malignant gliomas. Anticancer Res 1996; 16: 1251-1257.

17 Harrington K J, Linardakis E, Vile R G. Transcriptional control: an essential component of cancer gene therapy strategies? Adv Drug Deliv Rev 2000; 44: 167-184.

18 Horst M et al. Targeting malignant gliomas with a glial fibrillary acidic protein (GFAP)-selective oncolytic adenovirus. J Gene Med 2007; 9: 1071-1079.

19 John B et al. Human MicroRNA targets. PLoS Biol 2004; 2: e363.

20 Kloosterman W P, Plasterk R H. The diverse functions of microRNAs in animal development and disease. Dev Cell 2006; 11: 441-450.

21 Krichevsky A M et al. A microRNA array reveals extensive regulation of microRNAs during brain development. Rna 2003; 9: 1274-1281.

22 Landgraf P et al. A mammalian microRNA expression atlas based on small RNA library sequencing. Cell 2007; 129: 1401-1414.

23 Lewis B P, Burge C B, Bartel D P. Conserved seed pairing, often flanked by adenosines, indicates that thousands of human genes are microRNA targets. Cell 2005; 120: 15-20.

24 Lin S Y et al. The C elegans hunchback homolog, hbl-1, controls temporal patterning and is a probable microRNA target. Dev Cell 2003; 4: 639-650.

25 Lu J et al. MicroRNA expression profiles classify human cancers. Nature 2005; 435: 834-838.

26 Maron A et al. Differential toxicity of ganciclovir for rat neurons and astrocytes in primary culture following adenovirus-mediated transfer of the HSVtk gene. Gene Ther 1997; 4: 25-31.

27 McKie E A, Graham D I, Brown S M. Selective astrocytic transgene expression in vitro and in vivo from the GFAP promoter in a HSV RL1 null mutant vector-potential glioblastoma targeting. Gene Ther 1998; 5: 440-450.

28 Pulkkanen K J, Yla-Herttuala S. Gene therapy for malignant glioma: current clinical status. Mol Ther 2005; 12: 585-598.

29 Reinhart B J et al. The 21-nucleotide let-7 RNA regulates developmental timing in Caenorhabditis elegans. Nature 2000; 403: 901-906.

30 Reubinoff B E et al. Neural progenitors from human embryonic stem cells. Nat Biotechnol 2001; 19: 1134-1140.

31 Robson T, Hirst D G. Transcriptional Targeting in Cancer Gene Therapy. J Biomed Biotechnol 2003; 2003: 110-137.

32 Rosenfeld N et al. MicroRNAs accurately identify cancer tissue origin. Nat Biotechnol 2008; 26: 462-469.

33 Rutka J T et al. Role of glial filaments in cells and tumours of glial origin: a review. J Neurosurg 1997; 87: 420-430.

34 Saukkonen K, Hemminki A. Tissue-specific promoters for cancer gene therapy. Expert Opin Biol Ther 2004; 4: 683-696.

35 Sempere L F et al. Expression profiling of mammalian microRNAs uncovers a subset of brain-expressed microRNAs with possible roles in murine and human neuronal differentiation. Genome Biol 2004; 5: R13.

36 Shi R, Chiang V L. Facile means for quantifying microRNA expression by real-time PCR. Biotechniques 2005; 39: 519-525.

37 Stoff-Khalili M A et al. Cancer-specific targeting of a conditionally replicative adenovirus using mRNA translational control. Breast Cancer Res Treat 2008; 108: 43-55.

38 Wang C Y et al. Recombinant baculovirus containing the diphtheria toxin A gene for malignant glioma therapy. Cancer Res 2006; 66: 5798-5806.

39 Yang J, Su A I, Li W H. Gene expression evolves faster in narrowly than in broadly expressed mammalian genes. Mol Biol Evol 2005; 22: 2113-2118.

1. A nucleic acid molecule comprising:
a glial-specific promoter;
a coding sequence for a transgene; and
a plurality of miRNA target sites;
wherein each miRNA target site binds an miRNA that is down-regulated in a glioma cell compared to a normal glial cell, and wherein the glial-specific promoter and the plurality of miRNA target sites are both operably linked to the coding sequence for the transgene.

2. A nucleic acid molecule of claim 1, wherein the glial-specific promoter is an astrocyte-specific promoter.

3. A nucleic acid molecule of claim 2, wherein the glial-specific promoter is a glial fibrillary acidic protein promoter.

4. A nucleic acid molecule of claim 1, wherein the plurality of miRNA target sites comprises at least one has-miR-31, has-miR-127 or has-miR-143 target site.

5. A nucleic acid molecule of claim 4, wherein the plurality of miRNA target sites comprises at least one has-miR-31 target site, at least one has-miR-127 target site and at least one has-miR-143 target site.

6. A nucleic acid molecule of claim 5, wherein the plurality of miRNA target sites comprises at least two has-miR-31 target sites, at least two has-miR-127 target sites and at least two has-miR-143 target sites.

7. A nucleic acid molecule of claim 1, wherein the transgene encodes a gene product that induces direct killing of the glioma cell, an immunomodulation protein, a cytotoxin, an angiogenesis inhibitor protein, a tumour suppressor protein, a suicide protein, an apoptotic protein, an anti-angiogenic protein or an antibody.

8. A nucleic acid molecule of claim 1, wherein the transgene encodes HSV-tk or DT-A.

9. An expression vector comprising the nucleic acid molecule as defined in claim 1.

10. An expression vector of claim 9 that is a baculoviral vector.

11. A method of expressing a transgene in a glioma cell comprising:
transfecting a glioma cell with an expression vector as defined in claim 9.

12. A method of claim 11, wherein the glioma cell is an in vitro cell.

13. A method of claim 12, wherein the glioma cell is an ex vivo cell explanted from a subject.

14. A method of claim 11, wherein the glioma cell is an in vivo cell in a subject.

15. A transgenic cell comprising the expression vector as defined in claim 9.

16. A pharmaceutical composition comprising the expression vector as defined in claim 9.

17. A kit comprising the expression vector as defined in claim 9, and instructions for expressing a transgene in a glioma cell.
